(19) United States
(12) Patent Application Publication
(10) Pub. No.: US 2006/0206103 A1
(43) Pub. Date: Sep. 14, 2006

(54) Dermatological treatment device

(75) Inventors: Gregory B. Altshuler, Lincoln, MA (US); Andre V. Belikov, St. Petersburg (RU); Liam O'Shea, Medford, MA (US); Ilya Yaroslavsky, North Andover, MA (US); Andrei V. Erofeev, North Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 11/357,397

(22) Filed: Feb. 17, 2006

Related U.S. Application Data
(63) Continuation-in-part of application No. 10/080,652, filed on Feb. 22, 2002, now abandoned.
(60) Provisional application No. 60/272,745, filed on Mar. 2, 2001.
(60) Provisional application No. 60/654,130, filed on Feb. 18, 2005.

(51) Int. Cl. A61B 18/18

(52) U.S. Cl. 606/9

(57) ABSTRACT

A device and method for utilizing optical radiation to treat tissue are described. In one aspect, the device is a dermatological treatment device. The device can be used, for example, for treatment of dermatological and cosmetic conditions. The device can include a sensor that indicates when the device is in contact with a subject's tissue. Operation of the device can, in some instances, be partially or fully automated. The device can further include a light source that is air cooled and a cooling plate that is chilled preferably to 5° C. The device can also include a window that is enlarged to reduce the power density and facilitate heating of tissue at depth.

### RELATED APPLICATIONS

This application claims benefit of priority to U.S. Provisional Application No. 60/654,130, filed Feb. 18, 2005 entitled Dermatological Treatment Device. This application is a continuation-in-part of U.S. patent application Ser. No. 10/080,652 filed Feb. 22, 2002 entitled Apparatus and Method For Photocosmetic And Photodermatological Treatment, which claims the benefit of priority to U.S. Provisional Application No. 60/272,745, filed Mar. 2, 2001 entitled Apparatus and Method For Photocosmetic And Photodermatological Treatment. All contents disclosed in these applications is hereby incorporated by reference in its entirety.

### TECHNICAL FIELD

This invention relates generally to methods and apparatus for utilizing energy, e.g., optical radiation, to treat various dermatological and cosmetic conditions.

### BACKGROUND OF THE INVENTION

Fractional treatments generally have been directed to treating the epidermis, which is at the surface of skin tissue. However, for certain applications there is a need to provide treatments that extend further into the tissue.

Heating tissue at depth can be done with various wavelengths of EMR, both visible and non-visible. Infrared, also known as radiant heat, is a form of energy that heats objects directly through a process called conversion. Infrared radiation is emitted by any object that has a temperature (i.e. radiates heat). Infrared is not visible, but can be felt in the form of heat. The infrared segment of the electromagnetic spectrum occurs just below or “infra” to red light as the next lowest energy band of light.

### SUMMARY OF THE INVENTION

One aspect of the invention is a handheld dermatological device that includes a light source assembly that has a source for generating EMR and a cooling surface that defines a target treatment area on the tissue when located in proximity to the tissue. The light source assembly is configured to transmit EMR from the source, and through the cooling surface during operation. The devices also has first cooling mechanism for cooling the radiation source, and a second cooling mechanism for cooling the cooling surface.

Preferred embodiments of this aspect of the invention may include some of the following additional features. The dermatological treatment device can include a fan configured to pump air to cool the source, and a heatsink in thermal communication with the source. The fan pumps air over the heatsink to remove heat from the heatsink device during operation. The heatsink includes a plurality of cooling fins. The heatsink is thermally coupled to the source via a reflector, and the fan is configured to cool the source, the reflector, and the heatsink. The handheld dermatological device also has a control unit for controlling the first cooling mechanism. The control unit further includes a controller in electrical communication with a temperature sensor and in electrical communication with the fan, such that the controller can automatically control the first cooling mechanism based on information received from the temperature sensor.

The second cooling mechanism is a circulatory system for circulating a coolant that includes a chiller for cooling the tissue being treated to approximately at least 5° C. The second cooling mechanism also includes a pump, a cooling input, and a cooling output. The cooling input is connected to a cooling window at an input connection and the cooling output is connected to the cooling window at an output connection. The second cooling mechanism is configured to supply cooling fluid to the cooling window during operation via the cooling input and to extract heated coolant from the cooling window via the cooling output to cool the cooling window. The cooling mechanism further includes a chiller.

The second cooling mechanism also includes a temperature sensor for monitoring the temperature of the tissue and a control unit for controlling the second cooling mechanism. The control unit further comprises a controller in electrical communication with a temperature sensor and in electrical communication with the pump. The controller is configured to automatically control the pump based on information received from the temperature sensor.

Another aspect of the invention is a window of a dermatological treatment device that is configured to transmit EMR from a source of the device to tissue being treated. The window has a pane configured to allow EMR to pass from the dermatological treatment device to the tissue being treated. The window also has a first channel extending across substantially across a length of the pane and a frame extending about the pane to secure the pane in the dermatological treatment device. The window includes a first cooling input in fluid communication with a first end of the first channel and a first cooling output in fluid communication with a second end of the first channel. The window is configured to be cooled during operation by fluid traveling through the cooling input, through the first channel and out the second end of the first channel.

Preferred embodiments of this aspect of the invention may include some of the following additional features. The channel of the window is a groove having an open portion extending along a surface of the pane. The window also has an optical surface abutting the surface of the pane such that the groove is enclosed during operation to allow fluid to flow through the channel and to prevent the fluid from flowing out of the open portion. The window also has an optical material between the pane and the optical surface. The material allows some EMR to pass from the dermatological treatment device to the tissue being treated, and can be a dielectric coating.

Another aspect of the invention is a dermatological treatment device for treating tissue located at a depth of at least approximately 0.5 mm. The device includes a housing containing an EMR source and a window. The window is configured to transmit EMR from the source to the tissue being treated. The source is configured to produce at least 500 W of EMR and the window has an area sufficiently large to produce a power density of less than 5 W/cm².

Preferred embodiments of this aspect of the invention may include some of the following additional features. The pulse width of the power source is greater than or equal to 0.5 seconds and less than or equal to 600 seconds. The EMR source is configured to produce at least 1000 W.

Another aspect of the invention is an apparatus for performing a treatment on tissue, that includes a housing having a cooling surface that defines a target treatment area on the tissue when located in proximity to the tissue, a radiation source for generating EMR that passes through the cooling surface, and a sensor to indicate when the cooling surface is in proximity to the tissue.

Preferred embodiments of this aspect of the invention may include some of the following additional features. Activation of the sensor indicates that the cooling surface contacts the tissue. The sensor can be an e-field sensor, a capacitive sensor, a resistive sensor, a pressure sensor, or an H-field sensor. The sensor can be configured to detect changes in an electrical field.

The sensor is in electrical communication with a controller that is configured to provide signals in response to information obtained from the sensor. The controller issues a first signal corresponding to the detection by the sensor that no tissue is in close proximity and a second signal corresponding to the detection by the sensor that a first tissue is in close proximity. The controller issues a third signal corresponding to the detection by the sensor that a second tissue is in close proximity to the sensor. The controller distinguishes between tissue types based on the input from the sensor. The controller commands a first action in response to the detection of the first tissue type and a second action in response to the detection of the second tissue type. The first action is to treat the tissue. The second action is to not treat the tissue.

The sensor can include a first node and a second node disposed about the cooling surface. The nodes are in contact with the tissue when the cooling surface is in contact with the tissue and are not in contact with the tissue when the cooling surface is not completely in contact with the tissue. The sensor measures the current between the nodes when in contact with the skin. The sensor indicates that the skin is in contact with the sensor when a current is detected between the nodes.

The sensor can be mounted on the housing, and can be a microswitch. The device also may have an output device operably connected to the sensor. The output device is one of a visual device, an audio device, or a vibrating device. A feedback mechanism may also be connected to the sensor. The feedback mechanism indicates to an operator of the apparatus the amount of time the cooling surface is required to stay in contact with the tissue for safe operation. The feedback mechanism prevents firing of the radiation source if contact of the cooling surface with the tissue is broken. The feedback mechanism prevents firing of the radiation source until after a predetermined cooling time has elapsed.

The device also has a control unit to implement a preset cooling time before allowing firing of the radiation source. The control unit implements a preset firing time for the radiation source. The device can also be a handheld device, and the control unit can be operably coupled to the handheld device.

The radiation source can be a monochromatic source such as a laser. Alternatively, the radiation source can be a halogen lamp, a radiant lamp, an incandescent lamp, an arc lamp, and a fluorescent lamp.

The cooling surface can be made of a deformable or viscoelastic material, like a gel. The cooling surface can also be made of a solid material, such as glass, sapphire or plastic.

The device may have a contact frame that is operably coupled to the housing. The contact frame is movable from an extended position to a retracted position in which it is in proximity to the cooling surface. The sensor activates when the frame is in the retracted position. The sensor activates when the cooling surface is in proximity to the contact frame.

The contact frame has an interior portion that is open to allow passage of EMR. A push rod is connected to the contact frame and is operably coupled to the sensor, such that the push rod activates the sensor when the cooling surface contacts the contact frame. The sensor is mounted on one of the cooling surface and the contact frame.

Another aspect of the invention is an apparatus for performing a treatment on tissue that includes a housing having a means for cooling the tissue. The means for cooling the tissue includes a surface that defines a target treatment area on the tissue when located in proximity to the tissue. The housing also includes a means for generating EMR. The EMR passes through the surface during irradiation. The housing also includes a means for sensing contact of the means for cooling with the tissue.

Preferred embodiments of this aspect of the invention may include some of the following additional features. The means for sensing activates when the means for cooling contacts the contact frame. Activation of the means for sensing indicates that the means for cooling contacts the tissue. A contact frame is operably coupled to the housing. The contact frame is movable from an extended position to a position in which it is in contact with the means for cooling.

Another aspect of the invention is a method of operating a handheld dermatological device, which includes sensing contact of a cooling surface of the handheld device with tissue, indicating to a user of the handheld device when the cooling surface contacts the tissue, and automatically interrupting firing of a radiation source of the handheld device if the cooling surface loses contact with the tissue.

Preferred embodiments of this aspect of the invention may include some of the following additional features. The method can include sensing contact of the cooling surface with tissue, indicating to the user if the cooling surface loses contact with the tissue. The act of sensing contact comprises determining when a contact frame of the handheld device contacts the cooling surface. The contact of the contact frame with the cooling surface indicates contact of the cooling surface with the tissue.

The method may further include distinguishing a first tissue type in contact with the sensor from a second tissue type, and taking an action based on the tissue type. The act of taking an action includes not irradiating the tissue if the tissue corresponds to an untreatable tissue type and irradiating the tissue if the tissue corresponds to a treatable tissue type. The act of indicating to the user includes activating one of a visual indicator and an audio indicator.

Another aspect of the invention is a method of automatically operating a handheld dermatological device, which includes sensing contact of a cooling surface of the handheld device with tissue, instituting a preset cooling time for cooling of the tissue prior to irradiating the tissue with a radiation source of the handheld device, instituting a preset firing time of the radiation source after the preset cooling time, and interrupting firing of the radiation source if the cooling surface loses contact with the tissue.

Preferred embodiments of this aspect of the invention may include some of the following additional features. The method may further include indicating to the user if the cooling surface loses contact with the tissue, after sensing contact of the cooling surface with tissue. The act of indicating to the user includes activating one of a visual indicator and an audio indicator. The act of sensing contact comprises determining when a contact frame of the handheld device contacts the cooling surface, wherein contact of the contact frame with the cooling surface indicates contact of the cooling surface with the tissue.

### BRIEF DESCRIPTION OF THE DRAWINGS

Non-limiting embodiments of the present invention will be described by way of example with reference to the accompanying drawings in which:

FIG. 1 is a schematic diagram of one embodiment of the invention, shown in proximity to a tissue sample;

FIG. 2 is a side view of a schematic diagram of part of a handheld dermatological device according to one embodiment of the invention;

FIG. 3 is a second side view of the handheld dermatological device of FIG. 2;

FIG. 4 is a third side view of the handheld dermatological device of FIG. 2;

FIG. 5 is a fourth side view of the handheld dermatological device of FIG. 2;

FIG. 6 is a fifth side view of the handheld dermatological device of FIG. 2;

FIG. 7 is a sixth side view of the handheld dermatological device of FIG. 2;

FIG. 8 is a front view of the handheld dermatological device of FIG. 2;

FIG. 9 is a partial view from the front of a lamp, reflector, and optics of the handheld dermatological device of FIG. 2;

FIG. 10 is a perspective view of the handheld dermatological device of FIG. 2;

FIG. 11 is a second perspective view of the handheld dermatological device of FIG. 2;

FIG. 12 is a back view of the handheld dermatological device of FIG. 2;

FIG. 13 is a second back view of the handheld dermatological device of FIG. 2;

FIG. 14 is a bottom view of the handheld dermatological device of FIG. 2;

FIG. 15 is a side view of the housing structure and complete unit of the handheld dermatological device of FIG. 2;

FIG. 16 is a flow chart that illustrates the operation of one embodiment of the invention.

FIG. 17 is a graph showing the relationship between treatment time and the depth of heating for infrared radiation without pre-cooling the treated tissue; and

FIG. 18 is a graph showing the relationship between treatment time and surface skin temperature;

FIG. 19 is a side view of an alternative embodiment of a handheld dermatological device;

FIG. 20 is a cross-sectional side view of the handheld dermatological device of FIG. 19;

FIG. 21 is a schematic top view of a window for use in the handheld dermatological device of FIG. 19;

FIG. 22 is a schematic side view of the window of FIG. 21;

FIG. 23 is a schematic bottom view of an embodiment of a portion of the handheld dermatological device of FIG. 19;

FIGS. 24A and 24B are schematic side views of the portion of the handheld dermatological device shown in FIG. 23 during operation;

FIG. 25 is a schematic side view of an alternate embodiment for a window of a dermatological device;

FIG. 26 is a schematic side view of an alternate embodiment of a waveguide;

FIG. 27 is a bottom view of the waveguide of FIG. 26; and

FIG. 28 is a bottom view of an alternate embodiment of a face of a dermatological device.

### DETAILED DESCRIPTION

The benefits of being able to raise and/or lower the temperature in a selected region of tissue for various therapeutic and cosmetic purposes have been known for some time. For instance, heated pads or plates or various forms of electromagnetic radiation (EMR), including microwave radiation, electricity, infrared radiation, and ultrasound have previously been used for heating subdermal muscles, ligaments, bones and the like to, for example, increase blood flow, to otherwise promote the healing of various injuries and other damage, and for various therapeutic purposes, such as frostbite or hyperthermia treatment, treatment of poor blood circulation, physical therapy, stimulation of collagen, cellulite treatment, adrenergic stimulation, wound healing, psoriasis treatment, body reshaping, non-invasive wrinkle removal, etc. The heating of tissues has also been utilized as a potential treatment for removing cancers or other undesired growths, infections and the like. Heating may be applied over a small, localized area, over a larger area, for example to the hands or feet, or over larger regions of tissue, including the entire body.

Because most of the techniques described above involve applying energy to tissue at depth through the subject's skin surface, peak temperature generally occurs at or near the subject's skin surface and decreases, sometimes significantly, with depth. The radiation is both highly scattered and highly absorbed in surface layers of tissue, precluding significant portions of such radiation from reaching the tissue regions at depth to cause heating thereof. In view of the energy losses due to scattering and absorption, a substantial amount of optical (including near infrared) energy must be applied in order for enough energy to reach a region of tissues at depth to have a desired effect. However, such a high amount of optical energy can cause damage to the surface layers of tissue, making it difficult to achieve desired photothermal treatments in tissue regions at depth. For these reasons, optical radiation has heretofore had at most limited value for therapeutic and cosmetic treatments on tissue at depth.

Methods of deep heating are also desirable for fractional treatments, which depend, in part, upon the discovery that, when using EMR to treat tissues, there are substantial advantages to producing lattices of EMR-treated islets in the tissue rather than large, continuous regions of EMR-treated tissue. The lattices are periodic patterns of islets in one, two or three dimensions in which the islets correspond to local maxima of EMR-treatment of tissue. The islets are separated from each other by non-treated tissue. The EMR-treatment results in a lattice of EMR-treated islets which have been exposed to a particular wavelength or spectrum of EMR, and which is referred to herein as a lattice of “optical islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” By producing EMR-treated islets rather than continuous regions of EMR-treatment, more EMR energy can be delivered while lowering the risk of bulk tissue damage

To more effectively treat tissue with near infrared radiation, the skin at the surface of the tissue is typically cooled to a temperature of approximately 5° C., although other temperatures are used. Thus, the technique of the present invention combines advantageous features of non-ablative and fractional techniques.

Applications in which the invention may be useful include the treatment of various diseases and cosmetic enhancements, particularly, cellulite and subcutaneous fat treatment, physical therapy, muscle and skeletal treatments, including relief of pain and stiffness for muscles and joints, and treatment of spinal cord problems, and treatment of cumulative trauma disorders (CTD's) such as carpel tunnel syndrome (CTS), tendonitis and bursitis, fibromyalgia, lymphedema and cancer therapy and skin rejuvenation treatments, including, for example, skin smoothing, wrinkle and rhytide reduction, pore size reduction, skin lifting, improved tone and texture, stimulation of collagen production, shrinkage of collagen, reduction of skin dyschromia (i.e. pigment non-uniformities), reduction telangiectasia (i.e. vascular malformations), improvement in skin tensile properties (e.g. increase in elasticity, lifting, tightening), treatment of acne, hypertrophic scars, reducing body odor, removing warts and calluses, treating psoriasis, and decreasing body hair.

The present invention provides means for effective deep heating of tissue using both fractional and non-fractional procedures. For fractional procedures, the embodiments described below may create non-uniform (modulated) temperature profiles (MTP), including deep in the dermis and in hypodermis (typically, at depths exceeding 500 μm) or superficially in the epidermis and/or dermis. In some embodiments, such profiles result in formation of a pattern (lattice) of islets of damage (LID). Active or passive cooling can be applied to epidermal surface in order to prevent epidermal damage.

Creation of MTPs leads to improvements in skin structure and texture via the following mechanisms (the list is not exclusive):

1. Lifting and tightening of skin as a result of shrinkage of collagen fibrils subjected to elevated temperature.

2. Lifting and tightening of skin as a result of coagulation of localized areas in the dermis and hypodermis.

3. Improvement in skin texture as a result of coagulation of localized areas in the dermis and hypodermis.

4. Promotion of collagen production due to healing response to thermal stress and/or thermal shock.

A number of other local and systemic pathologies can be treated with the technique:

1. Cellulite: By changing mechanical stress distribution at the dermis/hypodermis border, the appearance of cellulite can be improved.

2. Acne: By selecting the wavelength of the optical radiation to promote preferential absorption of the optical energy by sebum and/or organizing the pattern to target preferentially sebaceous glands, selective destruction of the glands can be achieved.

3. Hypertrophic scars: By inducing tightening and shrinkage in the scar tissue, transformation of the abnormal connective tissue to normal one can be initiated.

4. Odor reduction: By selectively targeting eccrine glands, production of eccrine sweat can be reduced, and its composition can be changed.

5. Non-skin-surface texturing: The technique can be used for organ augmentation (e.g., lips).

One embodiment of the invention is a handheld dermatological device that incorporates a mechanism for cooling a subject's skin surface concurrently with the application of optical radiation thereto. While the radiation reaches the tissue at depth to be treated quickly to begin the heating thereof, cooling propagates as a cold wave, protecting tissue above the treatment region and moving the depth of maximum heating further into the skin. In one embodiment, the cooling wave can propagate to a depth just above the treatment region, but does not extend to the treatment region at least until sufficient energy has been delivered to the treatment region to effect the desired treatment. The cooling mechanism of the device can cool the subject's skin prior to, during, and/or after the application of radiation thereto to more effectively protect tissue above the treatment region and to insure that the maximum temperature rise in the irradiated tissue occurs at or near a desired depth. This may also permit higher energy and shorter duration of radiation pulses to be applied to the skin without any damage or minimal damage to tissue above the desired depth. The head used to apply the radiation may also be used to apply cooling. The handheld dermatological device can include a sensor mounted adjacent the cooling mechanism near the subject's skin. Such a sensor can indicate when the cooling mechanism contacts the subject's skin (or looses contact with the subject's skin), thus indicating to the user when it is safe to begin application of radiation.

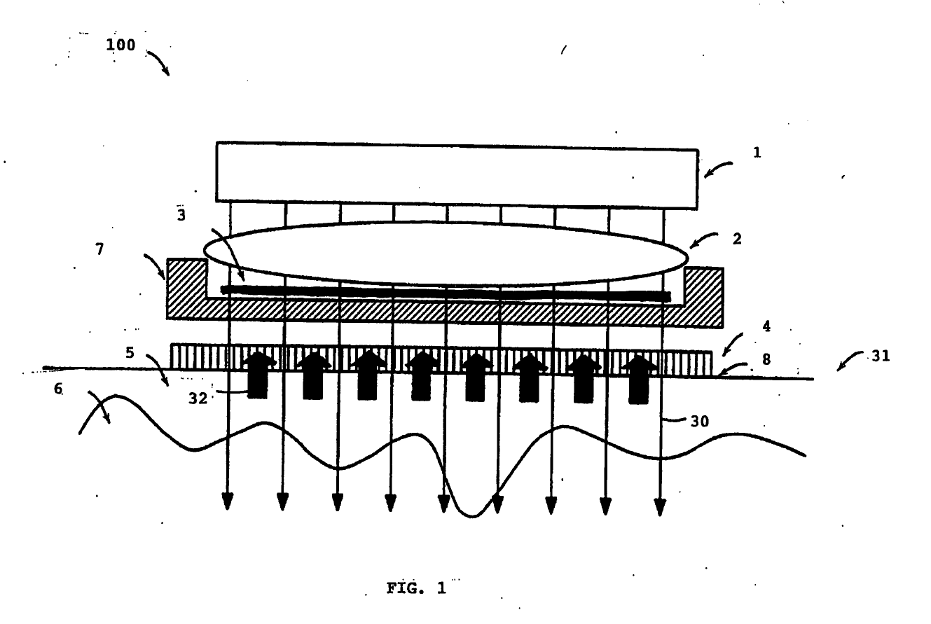

FIG. 1 shows an apparatus 100 according to one embodiment of the invention. For this apparatus, optical energy 30 from a suitable energy source 1 passes through optical (for example, focusing) device 2, filter 3, cooling mechanism 4 and contact plate 8, before reaching tissue 31 (i.e., the subject's skin). In some embodiments of the invention, certain of these components, such as, for example, filter 3 where a monochromatic energy source is utilized or optical device 2, may not necessarily be present. In other embodiments, the apparatus may not contact the skin. In yet another embodiment, there is no cooling mechanism 4 such that there is only passive cooling between the contact plate and the skin.

A suitable optical impedance matching lotion or other suitable substance would typically be applied between plate 8 and tissue 31 to provide enhanced optical and thermal contact. Tissue 31, as shown in FIG. 1, is divided into an upper region 5, which, for applications where radiation is applied to the skin surface, would be the epidermis and dermis, and a lower region 6, which would be a subdermal region in the previous example. Region 6, for instance, can be the hypodermis.

Energy 30, possibly in conjunction with one or a combination of focusing from optical device 2, and wavelength selection from filter 3, and with cooling from cooling mechanism 4, results in maximum heating occurring at a selected depth in tissue 31. The selected depth can be, as previously indicated, at or near the junction of regions 5 and 6 or in lower region 6, and it can also be in region 5 or in the hypodermis.

The energy source 1 may be any suitable electromagnetic radiation (EMR) source, but will preferably be a source emitting visible light, or energy in the near infrared and infrared ranges. The light sources used in conjunction with the invention may be coherent and non-coherent sources, able to produce optical energy at a desired wavelength or a desired wavelength band or in multiple wavelength bands. The exact energy source 1, and the exact energy chosen, may be a function of the type of treatment to be performed, the tissue to be heated, the depth within the tissue at which treatment is desired, and of the absorption of that energy in the desired area to be treated. Energy source 1 may produce EMR, such as near infrared or visible light radiation over a broad spectrum, over a limited spectrum, or at a single wavelength, such as would be produced by a light emitting diode or a laser. In certain cases, a narrow spectral source may be preferable, as the wavelength(s) produced by the energy source may be targeted towards a specific tissue type or may be adapted for reaching a selected depth. In other embodiments, a wide spectral source may be preferable, for example, in systems where the wavelength(s) to be applied to the tissue may change, for example, by applying different filters, depending on the application. Acoustic, RF or other EMF sources may also be employed in suitable applications.

For example, UV, violet, blue, green, yellow light or infrared radiation (e.g., about 290-600 nm, 1400-3000 nm) can be used for treatment of superficial targets, such as vascular and pigment lesions, fine wrinkles, skin texture and pores. Blue, green, yellow, red and near IR light in a range of about 450 to about 1300 nm can be used for treatment of a target at depths up to about 1 millimeter below the skin. Near infrared light in a range of about 800 to about 1400 nm, about 1500 to about 1800 nm or in a range of about 2050 nm to about 2350 nm can be used for treatment of deeper targets (e.g., up to about 3 millimeters beneath the skin surface). The following table shows examples of the wavelengths of electromagnetic energy that are thought to be suitable for treating various cosmetic and medical conditions.

TABLE 1
Uses of Light of Various Wavelengths In Photocosmetic Procedures

| Treatment condition or application | Wavelength of Light, nm |
|---|---|
| Anti-aging | 400-2700 |
| Superficial vascular | 290-600 |
| | 1300-2700 |
| Deep vascular | 500-1300 |
| Pigmented lesion, de pigmentation | 290-1300 |
| Skin texture, stretch mark, scar, porous | 290-2700 |
| Deep wrinkle, elasticity | 500-1350 |
| Skin lifting | 600-1350 |
| Acne | 290-700, |
| | 900-1850 |
| Psoriasis | 290-600 |
| Hair growth control | 400-1350 |
| PFB | 300-400, |
| | 450-1200 |
| Cellulite | 600-1350 |
| Skin cleaning | 290-700 |
| Odor | 290-1350 |
| Oiliness | 290-700, |
| | 900-1850 |
| Lotion delivery into the skin | 1200-20000 |
| Color lotion delivery into the skin | Spectrum of absorption of color |
| | center and 1200-20000 |
| Lotion with PDT effect on skin | Spectrum of absorption of photo |
| condition including anti cancer effect | sensitizer |
| ALA lotion with PDT effect on skin | 290-700 |
| condition including anti cancer effect | |
| Pain relief | 500-1350 |
| Muscular, joint treatment | 600-1350 |
| Blood, lymph, immune system | 290-1350 |
| Direct singlet oxygen generation | 1260-1280 |

The energy source 1 can be any variety of a coherent light source, such as a solid-state laser, dye laser, diode laser, fiber laser, or other coherent light source. For example, energy source 1 may be a radiant lamp, a halogen lamp, an incandescent lamp, an arc lamp, a fluorescent lamp, a light emitting diode, a laser (including diode and fiber lasers), the sun, or other suitable optical energy source. As another example, the energy source 1 can be a neodymium (Nd) laser, such as a Nd:YAG laser. In addition, multiple energy sources may be used which are identical or different. For example, multiple laser sources may be used and they may generate optical energy having the same wavelength or different wavelengths. As another example, multiple lamp sources may be used and they may be filtered to provide the same or different wavelength band or bands. In addition, different types of sources may be included in the same device, for example, mixing both lasers and lamps.

In this exemplary embodiment, the energy source 1 includes a neodymium (Nd) laser generating radiation having a wavelength around 1064 nm. Such a laser includes a lasing medium, e.g., in this embodiment a neodymium YAG laser rod (a YAG host crystal doped with Nd⁺³ ions), and associated optics (e.g., mirrors) that are coupled to the laser rod to form an optical cavity for generating lasing radiation. In other embodiments, other laser sources, such as chromium (Cr), Ytterbium (Yt) or diode lasers, or broadband sources, e.g., lamps, can be employed for generating the treatment radiation.

Lasers and other coherent light sources can be used to cover wavelengths within the 100 to 100,000 nm range. Examples of coherent energy sources are solid state, dye, fiber, and other types of lasers. For example, a solid state laser with lamp or diode pumping can be used. The wavelength generated by such a laser can be in the range of 400-3,500 nm. This range can be extended to 100-20,000 nm by using non-linear frequency converting. Solid state lasers can provide maximum flexibility with pulse width range from femtoseconds to a continuous wave.

Another example of a coherent source is a dye laser with non-coherent or coherent pumping, which provide wavelength-tunable light emission. Dye lasers can utilize a dye dissolved either in liquid or solid matrices. Typical tunable wavelength bands cover 400-1,200 nm and a laser bandwidth of about 0.1-10 nm. Mixtures of different dyes can provide multi wavelength emission. Dye laser conversion efficiency is about 0.1-1% for non-coherent pumping and up to about 80% with coherent pumping.

Another example of a coherent source is a fiber laser. Fiber lasers are active waveguides with a doped core or undoped core (Raman laser), with coherent or non-coherent pumping. Rare earth metal ions can be used as the doping material. The core and cladding materials can be quartz, glass or ceramic. The core diameter could be from microns to hundreds of microns. Pumping light could be launched into the core through the core facet or through cladding. The light conversion efficiency of such a fiber laser could be up to about 80% and the wavelength range can be from about 1,100 to 3,000 nm. A combination of different rare-earth ions, with or without additional Raman conversion, can provide simultaneous generation of different wavelengths, which could benefit treatment results. The range can be extended with the help of second harmonic generation (SHG) or optical parametric oscillator (OPO) optically connected to the fiber laser output. Fiber lasers can be combined into the bundle or can be used as a single fiber laser.

Diode lasers can be used for the 400-100,000 nm range. Since many photodermatology applications require a high-power light source, the configurations described below using diode laser bars can be based upon about 10-100 W, 1-cm-long, cw diode laser bar. Note that other sources (e.g., LEDs and microlasers) can be substituted in the configurations described for use with diode laser bars with suitable modifications to the optical and mechanical sub-systems.

Other types of lasers (e.g., gas, excimer, etc.) can also be used.

A variety of non-coherent sources of EMR (e.g., arc lamps, incandescence lamps, halogen lamps, light bulbs) can be used in the invention for the energy source 1. There are several monochromatic lamps available such as, for example, hollow cathode lamps (HCL) and electrodeless discharge lamps (EDL). HCL and EDL could generate emission lines from chemical elements. For example, sodium emits bright yellow light at 550 nm.

Linear arc lamps use a plasma of noble gases overheated by pulsed electrical discharge as a light source. Commonly used gases are xenon, krypton and their mixtures, in different proportions. The filling pressure can be from about several torr to thousands of torr. The lamp envelope for the linear flash lamp can be made from fused silica, doped silica or glass, or sapphire. The emission bandwidth is about 180-2,500 nm for clear envelope, and could be modified with a proper choice of dopant ions inside the lamp envelope, dielectric coatings on the lamp envelope, absorptive filters, fluorescent converters, or a suitable combination of these approaches.

In some embodiments, a Xenon-filled linear flash lamp with a trapezoidal concentrator made from BK7 glass can be used. As set forth in some embodiments below, the distal end of the optical train can, for example, be an array of microprisms attached to the output face of the concentrator. The spectral range of EMR generated by such a lamp can be about 300-2000 nm.

Incandescent lamps are one of the most common light sources and have an emission band from 300 to 4,000 nm at a filament temperature of about 2,500 C. The output emission can be concentrated on the target with reflectors and/or concentrators.

Halogen tungsten lamps utilize the halogen cycle to extend the lifetime of the lamp and permit it to operate at an elevated filament temperature (up to about 3,500 C), which greatly improves optical output. The emission band of such a lamp is in the range of about 300 to 3,000 nm.

Light-emitting diodes (LEDs) that emit light in the 290-2,000 nm range can be used to cover wavelengths not directly accessible by diode lasers.

Where optical device 2 is a focusing device, it may be any suitable device able to focus at least a portion of energy 30 arriving from energy source 1 at tissue 31, and in particular at a selected depth in tissue 31. For example, device 2 may include mirrors, prisms, reflectors, lenses such as Fresnel lenses, collimating lenses or focusing lenses, diffraction gratings, or other optical devices. Device 2 may also include a plurality or an array of devices listed above.

Filter 3 may be any suitable filter able to select, or at least partially select, certain wavelengths or wavelength bands from energy source 1. In certain embodiments, a specific set of wavelengths may be blocked by filter 3. It is also possible that undesired wavelengths in the energy from source 1 may be wavelength shifted in ways known in the art so as to enhance the energy available in the desired wavelength bands. Thus, filter 3 may include elements designed to absorb, reflect or alter certain wavelengths of electromagnetic radiation. For example, filter 3 may be used to remove certain types of wavelengths that are absorbed by surrounding tissues. For instance, dermis, hypodermis and epidermis tissues are primarily composed of water, as is much of the rest of the human body. By using a filter that selectively removes wavelengths that excite water molecules, the absorption of these wavelengths by the body may be greatly reduced, which may contribute to a reduction in the amount of heat generated by light absorption in these molecules. Thus, by passing radiation through a water-based filter, those frequencies of radiation that may excite water molecules will be absorbed in the water filter, and will not be transmitted into tissue 31. Thus, a water-based filter may be used to decrease the amount of radiation absorbed in tissue above the treatment region and converted into heat. For other treatments, absorption of the radiation by water may be desired or required for treatment.

FIG. 1 shows a cooling mechanism 4 adjacent to the surface of tissue 31. Cooling mechanism 4 may be any suitable cooling mechanism able to reduce the temperature of tissue 31. Heat energy 32 may be drawn from tissue 31 across contact plate 8 into cooling mechanism 4. For example, cooling mechanism 4 may be air or other suitable gas that is blown over contact plate 8, cooling water, or a cooling oil or other fluid. Mixtures of these substances, such as an oil and water mixture, may also be envisioned. Cooling mechanism 4 may have any suitable configuration, for example, a flat plate, a series of conducting pipes, a sheathing blanket, or a series of channels for the passage of air, or other gases, or liquid across plate 8. For example, in one embodiment, cooling mechanism 4 may be a water-cooled contact plate. In another embodiment, cooling mechanism 4 may be a series of channels carrying a coolant fluid or a refrigerant fluid (for example, a cryogen), which channels are in contact with tissue 31 or with plate 8. In yet another embodiment, cooling mechanism 4 may comprise a water or refrigerant fluid (for example R134A) spray, a cool air spray or air flow across the surface of tissue 31. In other embodiments, cooling may be accomplished through chemical reactions (for example, endothermic reactions), or through electronic cooling, such as Peltier cooling. In yet other embodiments, cooling mechanism 4 may have more than one type of coolant, or cooling mechanism 4 and/or contact plate 8 may be absent, for example, in embodiments where the tissue is cooled passively or directly, for example, through a cryogenic or other suitable spray. Sensors or other monitoring devices may also be embedded in cooling mechanism 4, for example, to monitor the temperature, or determine the degree of cooling required by tissue 31, and be manually or electronically controlled.

In certain cases, cooling mechanism 4 may be used to maintain the surface temperature of tissue 31 at its normal temperature, which may be, for example, 37 or 32° C., depending on the type of tissue being heated. In other embodiments, cooling mechanism 4 may be used to decrease the temperature of the surface of tissue 31 to a temperature below the normal temperature of that type of tissue. For example, cooling mechanism 4 may be able to decrease the surface temperature of tissue 31 to, for example, a range between 25° C. and −5° C.

In some embodiments of the invention, such as shown in FIG. 1, energy 30 from energy source 1 may pass through cooling mechanism 4. In these types of configurations, cooling mechanism 4 may be made from materials able to transmit at least a portion of energy 30, for example, air, water or other gases or fluids, glass, or a clear plastic. In other embodiments, cooling mechanism 4 may be formed out of a series of discrete channels, and energy 30 may pass between these channels. In other embodiments of the invention, energy 30 may not be directed through cooling mechanism 4.

Contact plate 8 may be made out of a suitable heat transfer material, and also, where the plate contacts tissue 31, of a material having a good optical match with the tissue. Sapphire is an example of a suitable material for plate 8. In some embodiments, contact plate 8 may have a high degree of thermal conductivity, for example, to allow cooling of the surface of the tissue by cooling mechanism 4. In other embodiments, contact plate 8 may be an integral part of cooling mechanism 4, or be absent. Contact plate 8 may be made out of a deformable or viscoelastic material in some embodiments of the invention, for example, a gel such as a hydrogel. In other embodiments, contact plate 8 may be made of a solid material, such as a glass, a crystal such as sapphire, or a plastic. In some embodiments of the invention, such as shown in FIG. 1, energy 30 from energy source 1, or a fraction thereof, may pass through contact plate 8. In these configurations, contact plate 8 may be made out of materials able to transmit at least a portion of energy 30, for example glass, sapphire, or a clear plastic, or contact plate 8 may be made in such a way as to allow at least a portion of energy 30 to pass through contact plate 8, for example, via a series of holes, passages, lenses, etc. within contact plate 8.

In some embodiments of the invention, energy source 1, optical device 2 and/or filter 3 may also require a cooling mechanism. This cooling mechanism may or may not be the same as the cooling mechanism 4 that cools tissue 31 through contact plate 8, as indicated by arrows 32 in FIG. 1. For example, in the embodiment shown in FIG. 1, cooling mechanism 7, shown separately from cooling mechanism 4, is used to cool filter 3 and/or optical device 2. The design of cooling mechanism 7 may be a function of the components used in the construction of the apparatus. In FIG. 1, cooling mechanism 7 and cooling mechanism 4 are illustrated as separate systems. However, in other embodiments, cooling mechanism 7 and cooling mechanism 4 may be part of the same system, or one or both may be absent. Cooling mechanism 7 may be any suitable cooling mechanism known in the art, such as air, water, or oil. Mixtures of these substances, such as an oil and water mixture, may also be envisioned. Cooling of the components may be accomplished through convective or conductive cooling.

One or more of energy source 1, optical device 2, filter 3, cooling mechanism 4, or cooling mechanism 7 may be electronically controlled. For example, sensors embedded in cooling mechanism 4 or contact plate 8 may determine the amount of energy reaching tissue 31, and may direct energy source 1 to produce more or less energy or may direct cooling mechanism 4 to increase or decrease cooling, depending on the application. Other sensors and the like may be embedded in any of the components illustrated herein. The controls may be, for example, electronically preprogrammed, or manually operable.

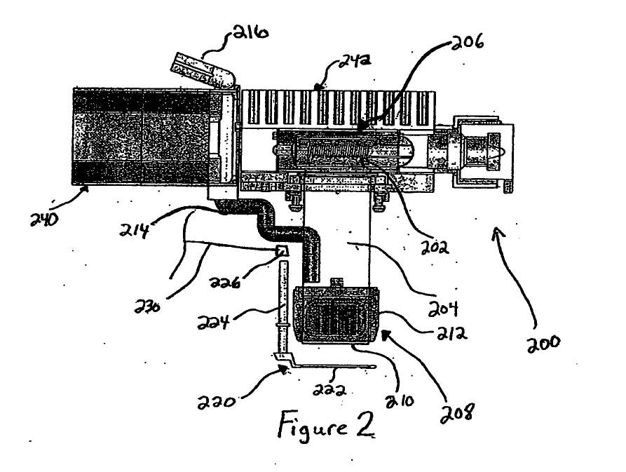

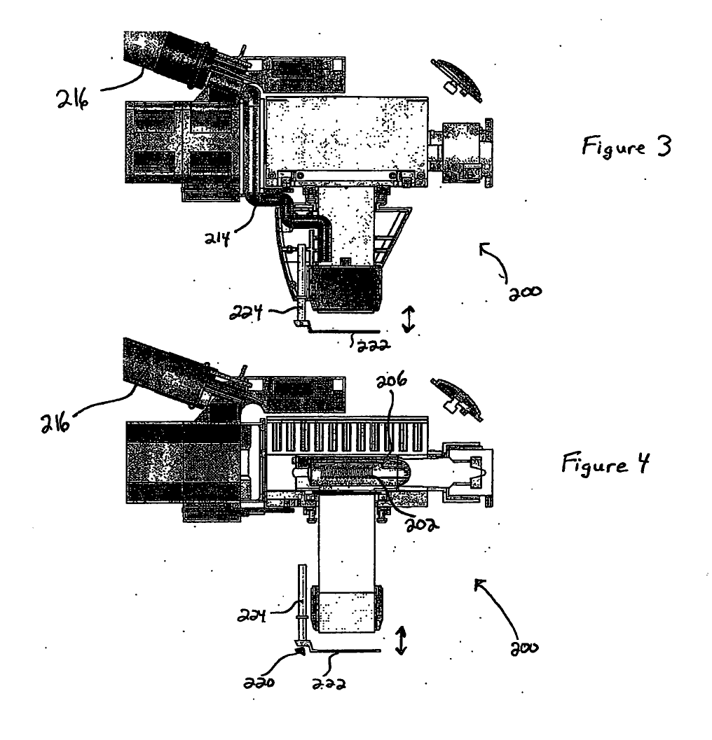

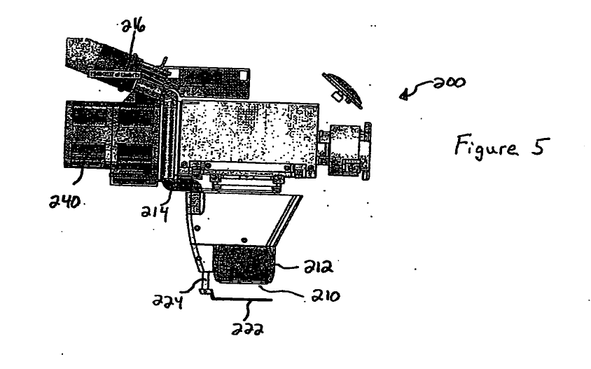

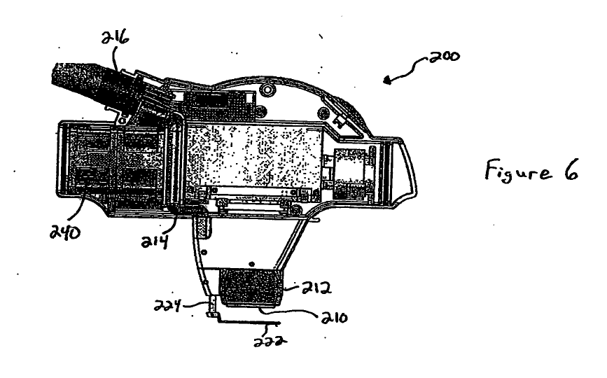

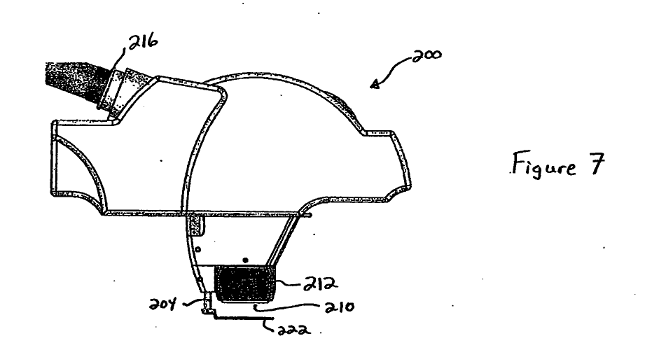

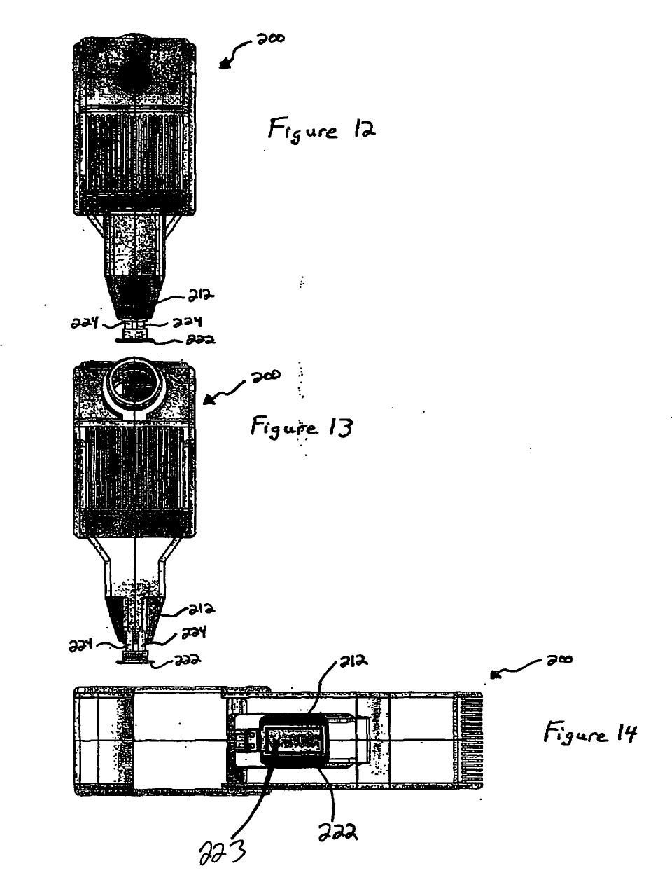

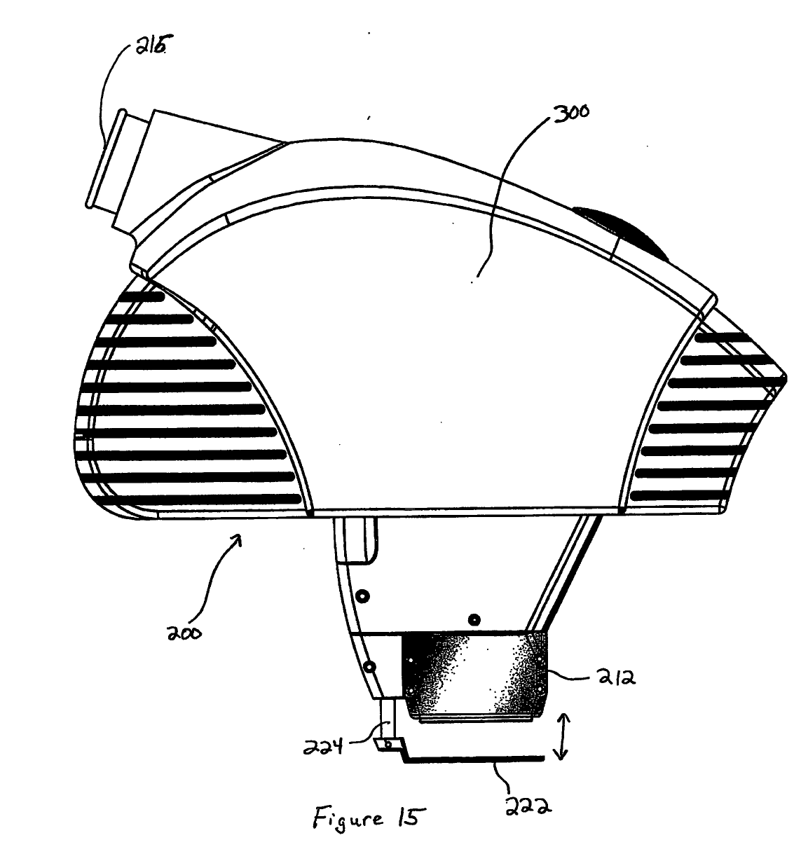

FIG. 2 is a side cross-sectional view of the handheld dermatological device 200 according to this embodiment of the invention. FIG. 2 illustrates most of the components of one embodiment of the handheld dermatological device 200. FIG. 15, on the other hand, is a side view of the complete handheld dermatological device 200, in a housing 300, according to one embodiment of the invention. FIGS. 3-14 are views of the handheld dermatological device 200 of FIG. 2 from varying angles, and these figures illustrate embodiments of the handheld dermatological device 200 in different states of construction. That is, FIGS. 3-14 do not depict the entire handheld dermatological device 200, including all of its components, in its housing 300.

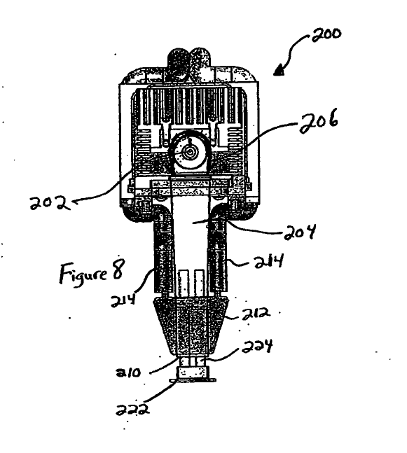

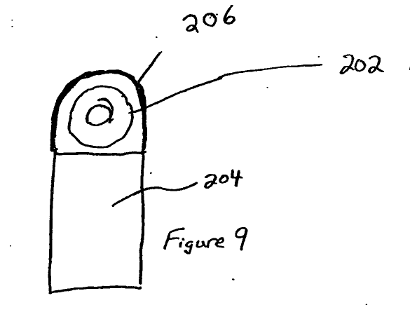

In the embodiment of FIGS. 2-15, a handheld dermatological device 200 includes many of the features discussed above in connection with FIG. 1. Referring to FIG. 2, the device 200 includes an energy source 202, which may be any suitable optical energy source able to produce optical energy at a wavelength that produces heating within tissue at the depth of a desired treatment region. In the embodiment of FIG. 2, the energy source 202 is, for example, a tungsten halogen lamp. Disposed above and in surrounding relation to the energy source 202 is a reflector 206. The reflector 206 serves to reflect energy from the energy source 202 (e.g. downward) toward skin contact plate 210. In other embodiments of the invention, such a reflector 206 is not used. In the embodiment of FIGS. 2, 8, and 9, the reflector 206 approximately semi-circular in cross-section (FIGS. 8, 9) and has a tubular length (FIG. 2). The reflector 206 can be made from any material known to reflect radiation, such as, for example, a metal. Preferably, the surface of reflector 206 is gold, although any highly reflective metal can be used, including silver or copper.

Disposed between the energy source 202 and the skin contact plate 210 in the embodiment of FIG. 2 is an optical device 204 and/or a filter (not shown). The optical device 204 can be a focusing device to focus at least a portion of energy from energy source 202 at tissue disposed below the device 200, and in particular at a selected depth in tissue. Optical device 204 may also be a waveguide, preferably made of quartz. The filter, if used, can be any suitable filter able to select, or at least partially select, certain wavelengths or wavelength bands from energy source 202. The optical device 204 and the filter, if used, can be the same as those discussed above in connection with the embodiment of FIG. 1.

In the embodiment of FIGS. 2-15, the handheld device 200 includes a cooling mechanism 208 disposed at a distal tip for application to the subject's skin or tissue. Such a cooling mechanism 208 can include a contact plate 210 to contact the subject's skin and a jacket 212 to hold the contact plate 210. The contact plate 210 can be made out of a suitable heat transfer material, such as those set forth above. The contact plate 210 can allow the radiation from the energy source 202 to pass through it in order to irradiate the subject's skin. In other embodiments, a mask, screen or shield (not shown), incorporated within or disposed above or below the contact plate 210 within the device 200, can block some of the radiation from reaching the subject's skin, thus creating selected areas of treatment on the subject's skin. In still other embodiments, an array of focusing elements (e.g., lenses, prisms) can be incorporated within or disposed above or below the contact plate 210 within the device 200 to focus or disperse the radiation to certain locations in the skin, thus creating selected areas of treatment on the subject's skin. (A further description of such methods and apparatus are disclosed in U.S. Pat. No. 6,997,923, issued Feb. 14, 2006 and assigned to Palomar Medical Technologies, Inc. U.S. Pat. No. 6,997,923 is incorporated herein by reference.)

In one embodiment, the contact plate 210 is made from sapphire. The cooling mechanism 208 can also include a jacket 212 disposed at the tip of the device 200 to hold the contact plate 210. In one embodiment, the jacket 212 can be a metal structure disposed around the contact plate 210. The jacket 212 can have an opening through its middle to allow for passage of radiation through the jacket 212. In the embodiment of FIGS. 2-15, the jacket 212 is configured to receive a coolant, such as water, air, or oil, which can circulate within the jacket 212 to remove heat from the jacket 212 and contact plate 210. The device 200 of FIGS. 2-15 also includes a cooling manifold 214 to supply coolant to the jacket 212. Alternatively, optical device 204 can be a waveguide which passes through jacket 212 such that one end of the waveguide provides contact surface 210. In use, the contact plate 210 defines the target treatment area on the subject's tissue.

The handheld device 200 can include a sensing mechanism 220 to indicate when the contact plate 210 contacts the subject's skin. The sensing mechanism 220 includes a contact frame 222, push rods 224, and a sensor 226. Sensor 226 can, for example, be a micro-switch. FIGS. 2-15 illustrate an embodiment of the invention, which incorporates a sensing mechanism 220 to sense contact of the cooling mechanism to the subject's skin. Sensing mechanism 220 is mounted adjacent the cooling mechanism and near the subject's skin. Such a sensing mechanism 220 can indicate when the cooling mechanism contacts the subject's skin and/or when the cooling mechanism looses contact with the subject's skin. Such a sensing mechanism 220 can also, in one embodiment, be incorporated within the apparatus 100 of FIG. 1.

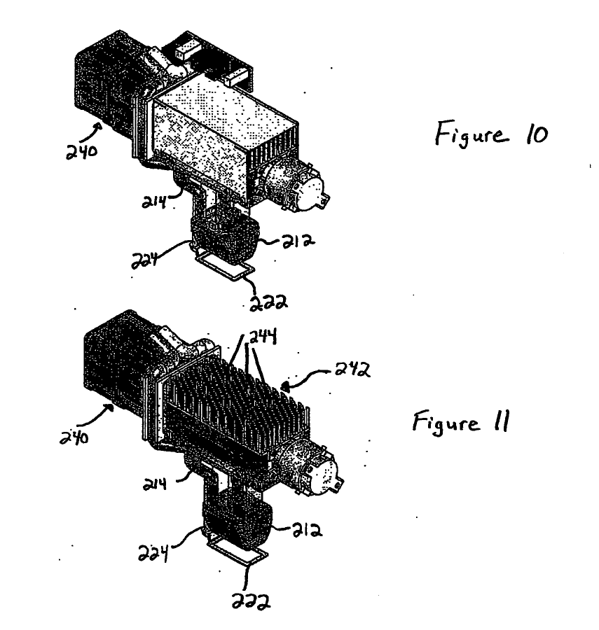

The contact frame 222 can have a rectangular cross-section, as shown in the embodiment of FIGS. 10-11. In other embodiments, the contact frame 222 can have a square or circular cross-section, or any other desired shape. As shown in FIGS. 10-11, the contact frame 222 can be shaped as a frame so that an interior portion of the frame 222 is open. Thus, radiation from the energy source 202 can be applied to the subject's skin through the interior portion of the contact frame 222. The contact frame 222 can be made from metal, plastic, or any other suitable materials.

The sensor 226 is a device that senses when the contact surface 210 touches the subject's skin. More particularly, the sensor 226 senses when the contact frame 222 touches the contact surface 210 of the cooling mechanism 208, which indicates that the contact surface 210 is in contact with the subject's skin. The sensor 226 can be any mechanical, optical, electro-optical, or other sensor that indicates contact of the contact surface 210 to the subject's skin. In one embodiment, the sensor 226 can be a micro-switch. The sensor 226 can be calibrated so that it is activated when the contact surface 210 touches the contact frame 222.

In the embodiment of FIGS. 2-15, the push rods 224 operably connect the contact frame 222 to the sensor 226. In the illustrative embodiment, two push rods 224 are connected to the contact frame 222. In this embodiment, both push rods 224 connect to one side of the contact frame 222. In other embodiments, the push rods 224 can be disposed on different sides of the contact frame 222. In other embodiments, only a single push rod 224 can be used. In still other embodiments, more than two push rods 224 can be used. In the embodiment of FIGS. 2-15, the push rods 224 contact the sensor 226, activating it, when the contact frame 222 contacts the contact surface 210 of the cooling mechanism 208.

The contact frame 222, push rods 224, and sensor 226 of the contact mechanism 220 can be operably connected to the device 200. In the illustrative embodiment of FIGS. 2-15, for example, the contact frame 222 is connected to the push rods 224, which in turn are connected through housing 300 and links (not shown) to the lower portion of the device 200. Such a link or links secures the push rods 224, and therefore also the contact frame 222, to the device 200, while allowing the push rods 224 and contact frame 222 to move up and down with respect to the device 200. As shown in FIGS. 3, 4, and 15 by a double-headed arrow, the contact frame 222 can move up and down with respect to the contact plate 210. The sensor 226 can, in one embodiment, be securely mounted to a housing 300 of the device 200. In another embodiment, sensor 226 can be located between the contact frame 222 and the contact plate 210, by being securely mounted to the contact frame 222 or the contact plate 210. In this embodiment, the sensor 226 is activated upon contact of the contact plate 210 with the contact frame 222. The contact mechanism 220 can also include, in some embodiments, a spring or other device to bias the contact frame 222 away from the contact plate 210 of the cooling mechanism 208.

In another embodiment of the invention, the sensor 226 can provide feedback to the user to indicate contact of the cooling plate 210, or the lack of such contact, with the subject's skin. In one embodiment, the sensor 226 can have an output on the handheld device 200. For example, the handheld device 200 can include a visual indicator, such as a light, that indicates when the contact plate 210 is in contact with the subject's skin. For instance, if the light is on, that can indicate that the contact plate 210 is in contact with the subject's skin, and if the light is off, that can indicate that the contact plate 210 is not in contact with the subject's skin. The handheld device 200 can, in other embodiments, include a speaker or other audio device to communicate to the user that the contact plate 210 is in contact with the subject's skin. The audio device can, in one embodiment, beep to indicate contact with the skin. In addition, the audio device can beep to indicate that contact of the cooling plate 210 with the skin has ended. In another embodiment, the audio device can produce a continuous tone during the entire period in which the contact plate 210 is in contact with the subject's skin. When the contact with the skin is broken, for instance, the sound can end. In another embodiment, tactile feedback can be provided to the user, for example, the handheld device 200 may vibrate when the contact plate 210 is in contact with the subject's skin.

In another embodiment, the sensor 226 of the sensing mechanism 220 can be electrically or optically connected through the cable (of connector 216) to the control unit (not shown). FIG. 2, for instance, depicts a wire 230 or cord that is connected at one end to the sensor 226. The other end of this wire 230 can be connected to the control unit through the connector 216. Thus, a visual and/or audio and/or tactile indicator, similar to those described above, can be produced at the control unit to indicate contact (or the lack thereof) of the cooling mechanism 208 with the subject's skin.

In one embodiment, the handheld device 200 of FIGS. 2-15 includes a connection 216 (FIGS. 3, 4) for an umbilical cord or cable connection to a control or base unit (not shown) that can communicate through control signals with the handheld device 200. The control unit can include, for example, a supply of coolant for the cooling mechanism 208. FIG. 2, for instance, depicts the cooling manifold 214 connecting the jacket 212 to the connection 216 for the umbilical cord. In another embodiment, the control unit can include power settings and the like for the energy source 202 within the handheld device 200. In addition, the control unit can include a microcomputer and/or a controller to control certain features of the invention, as will be described below in greater detail. The cable connecting the control unit to the connection 216 of the handheld device 200 can include supply lines for coolant and wires for control and power of the handheld device 200. In other embodiments, such a connection 216 might not be used.

Another embodiment of the invention is an air cooling mechanism and process for the handheld device 200. Referring to FIGS. 2-15, and more particularly to FIGS. 10-11, one example of an air cooling mechanism includes a fan 240 and a manifold 242. In one embodiment, the fan 240 can be an electrical fan supplied with power through the cable from the control unit. In addition, in some embodiments, the power (i.e., speed) of the fan can be controlled through the control unit. Any type of fan 240 can be used within the scope of the invention. In the embodiment of FIGS. 2-15, the fan 240 is compact enough to fit within the housing 300 of the handheld device 200.

In the embodiment of FIGS. 2-15, a manifold 242 surrounds the items within the handheld device 200 that require cooling. For instance, the energy source 202 and the reflector 206 may require cooling. In addition, numerous other parts within the device 200 might require cooling, such as the optical device 204, electrodes, and/or other reflecting surfaces within the device 200. The manifold 242 can be configured to supply cooling to such areas.

In the embodiment of FIGS. 2-15, the manifold 242 includes a plurality of fins 244. These fins 244 increase the cooling surface area of the manifold 242, which increases the cooling capacity of the device 200. The manifold 242 can be made from metal or any other suitable material. In addition to or in place of the fins 244, the manifold 242 can include one or more radiators of different types that aid in removing heat from the device 200. The manifold 242 can also include fins 244 or radiators that extend near any of the structures that require cooling. The fins 244 can extend in any direction, including upward as shown in FIGS. 10-11.

The fan 240 blows air through the manifold 242, removing heat from the manifold 242 and causing the device 200 to stay cool. With the incorporation of a fan 240 of sufficiently small size and sufficiently high power, such a cooling mechanism can efficiently remove heat from the handheld device 200 in a cost effective manner, without sacrificing size.

The embodiment of the invention depicted in FIGS. 2-15 uses air cooling for the energy source 202 and reflector 206, and it uses water cooling for the cooling mechanism 208 for contact with the subject's skin. In other embodiments, air cooling can also be used for the cooling mechanism 208. In addition, in such an embodiment, the cooling mechanism 208 can be part of the manifold 242.

When a halogen lamp is used as the energy source 202, the change in temperature is so great that air cooling through one or more small, inexpensive fans can be sufficient for the halogen lamp and reflectors of the device. Because generally the surface of the skin is required to be cooled to a much lower temperature, it is still preferable to cool the contact plate 210 (or cooling mechanism 208) with a coolant, such as a chilled fluid or gas. Use of a small fan to cool the lamp reduces the amount of coolant coming into the handheld device 200 from the control unit. This reduces the size of the umbilical cord required to carry coolant and the size and cost of the chiller required to cool the coolant.

During operation, a user applies the device 200 to a subject's skin. The user aligns the contact frame 222 around the precise area of the subject's skin that the user wants to treat. The operator then pushes down (or towards the skin surface) on the handheld device 200, causing the push rods 224 to extend upward within handheld device 200, to bring skin contact plate 210 into contact with the skin surface. When the user presses down or toward the skin on the handheld device 200, the contact plate 210 of the device 200 approaches the contact frame 222 and skin. In other words, as the user presses down on the handheld device 200, the contact frame 222 is pressed against the subject's skin and the push rods 224 move into the housing 300 as the contact plate 210 is forced toward contact frame 222 and skin. When skin contact plate 210 is in contact with the skin surface, push rods 224 activate sensor 226, which indicates such contact to the control unit and/or to the user of the handheld device.

Eventually, when the contact frame 222 comes into contact with the contact plate 210, the push rods 224 contact and activate the sensor 226, indicating that the contact plate 210 is in contact with the subject's skin. Because contact plate 210 is cooled, activation of the sensor 226 indicates that cooling of the skin has begun. The description above describes, and FIGS. 2-15 depict, one embodiment of a sensing mechanism 220. Other sensing mechanisms can also be used within the scope of the invention.

The use of a sensing mechanism 220 aids the user of the handheld device 200. For instance, if the user desires to cool the subject's skin prior to application of radiation, the sensing mechanism 220 aids the user of the handheld device in determining when the cooling mechanism 208 of the device 200 is in contact with the subject's skin. This prevents the user from accidentally believing that the cooling plate 210 is in contact with the subject's skin when it is, in fact, not in contact. Thus, in this embodiment, the sensing mechanism 220 can provide a safety feature for the device 200.

Once the user receives feedback indicating that the contact plate 210 is in contact with the skin, the user may fire the device 200 to irradiate the skin. Where pre-cooling is desired, the feedback from the sensor 226 indicating contact with the skin may be different for a pre-cooling time and may change to indicate to the operator that application of radiation can begin. For example, the feedback may provide a beeping sound while the device 200 is pre-cooling the skin and a continuous tone when it is safe for the user to fire the device 200 to irradiate the skin. In one embodiment, the device 200 may prevent firing by the user until the pre-cooling time is met, and if contact with the skin is broken, the device 200 may start the cycle over. In another embodiment, the firing time of the device 200 is preset such that once the user initiates firing, the device 200 will irradiate the skin for that preset time. In another embodiment, the device 200 will stop the radiation if contact with the skin surface is broken. In another embodiment, the device will provide feedback to the user after irradiation to indicate a post irradiation cooling time.

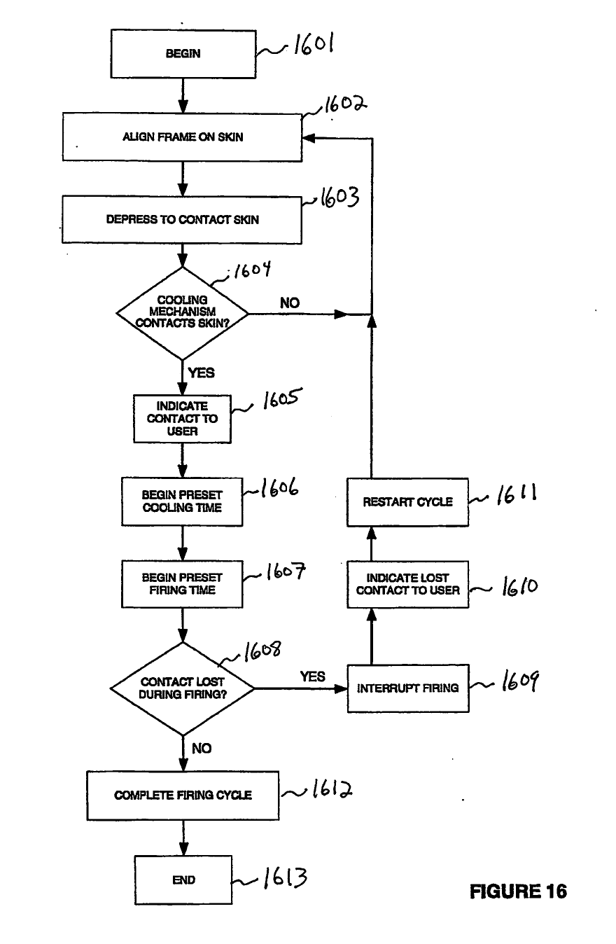

FIG. 16 is a flow chart, according to one embodiment of the invention, that illustrates how the device 200 and a control unit can work during operation to aid the user in radiating the subject's skin. The first three steps shown in FIG. 16 can be steps performed by the user. The remainder of the steps, in the embodiment of FIG. 16, can be automatically performed by the device 200 and control unit. In other embodiments, some of the steps can be automated and others can be performed by the user. First, at blocks 1601 and 1602, as set forth above, the user begins the procedure and aligns the contact frame 222 around the target area of the subject's skin. The user next depresses the device 200 against the subject's skin (at block 1603) until the sensor 226 indicates that the contact plate 210 contacts the skin in order to cool the skin. At block 1604, the device determines whether the contact plate 210 contacts the skin. When the sensor 226 indicates that the contact plate 210 touches the subject's skin, an indication is sent to the user indicating that such contact exists (at block 1605). If the device 200 or control unit do not provide such an indication, in one embodiment, the user should begin the process again.

In one embodiment, as illustrated in FIG. 16, at block 1606, the control unit and/or handheld device 200 can be configured with a preset cooling time. Such a preset cooling time is an amount of time that the device 200 will wait (or must wait), while the cooling mechanism contacts the subject's skin, before firing of the radiation. Such a preset cooling time can be used as a safety mechanism and/or as a method of automating treatment.

In some embodiments, as illustrated in FIG. 16, at block 1607, the control unit and/or handheld device 200 can be configured with a preset firing time of the energy source 202. Such a preset firing time is an amount of time that the energy source 202 will fire in order to radiate the subject's skin. Alternatively, such a preset firing time can be the number of firing cycles or pulses for the energy source 202 or some combination of the number of firing cycles and length of pulses of the radiation. Such a preset firing time can be used as a safety mechanism and/or as a method of automating treatment. Further, the combination of the use of a preset cooling time and preset firing time can be used to create an automated process. Different preset cooling times and preset firing times can be used for different treatments.

In another embodiment of the invention, as illustrated in FIG. 16, at block 1608, the sensor 226 can determine when contact of the cooling plate 222 with the skin is lost during treatment. As shown at block 1609 in FIG. 16, the control unit and/or handheld device 200 can be provided with an automatic interrupt if the sensor 226 indicates that contact of the contact plate 222 of the cooling mechanism 208 with the subject's skin has been lost. Such an automated interrupt provides a safety mechanism so that the subject's skin is not damaged, for example, by excess heat and/or irradiation. In such an embodiment, if the sensor 226 indicates that contact has been lost, an interrupt signal can shut off the energy source 202. Such an interrupt signal can be generated by the control unit. In another embodiment, the interrupt signal can be generated on the handheld device 200 so that firing of the energy source 202 is automatically interrupted if contact of the cooling mechanism 208 with the subject's skin is lost. In addition, as shown at block 1610 of FIG. 16, the control unit and/or handheld device 200 can provide an indication to the user that contact has been lost and firing has been interrupted. The user can then restart (block 1611) or abandon the process. In an alternative embodiment, such an automatic interrupt is not used. Instead, in such an embodiment, the control unit or handheld device 200 can indicate to the user that contact of the contact plate 222 with the subject's skin has been lost. In such an embodiment, the use of the device 200 can continue to fire the energy source 202, if desired, after contact of the cooling mechanism 208 with the subject's skin ends.

When a cycle of cooling and firing of radiation has been completed, irradiation of the tissues can end (block 1612) and the cycle can end (block 1613). The control unit and/or handheld device 200 can indicate to the user (through either a visual, audio or tactile signal) that it is safe to reposition the device 200 in order to begin another cycle on a different target area on the subject's skin.

As set forth above, many uses require cooling of the target area of the subject's skin prior to application of radiation. This can effectively protect tissue above the treatment region, can allow for higher fluences and shorter pulse durations, and can insure that the maximum temperature rise in the tissue occurs at or near a desired depth. Pre-cooling is preferable for certain applications, such as the treatment of cellulite, where light or other EMR is applied for a longer period to achieve heating at greater depths. In addition, application of cooling while the radiation is being applied to the subject's skin is necessary or desired for certain applications. Further, post cooling may be preferable in certain applications, for example, to dissipate following applications of light during vein treatments.

The time of radiation application may vary from approximately 2 seconds to approximately 2 hours for depths of approximately 1 mm to 50 mm, respectively. Depending on depth, the treatment being performed, and other factors, the power density may vary from approximately 0.2 to 50 W/cm², more preferably from approximately 0.5 to 20 W/cm², and most preferably from 0.5 to 10 W/cm or 0.5 to 5 W/cm². The handheld device 200 and/or control unit can have such radiation application times and power densities preset for different applications, as described above in connection with FIG. 16. In addition, different preset cooling times can be used in connection with different radiation application times and/or power densities.

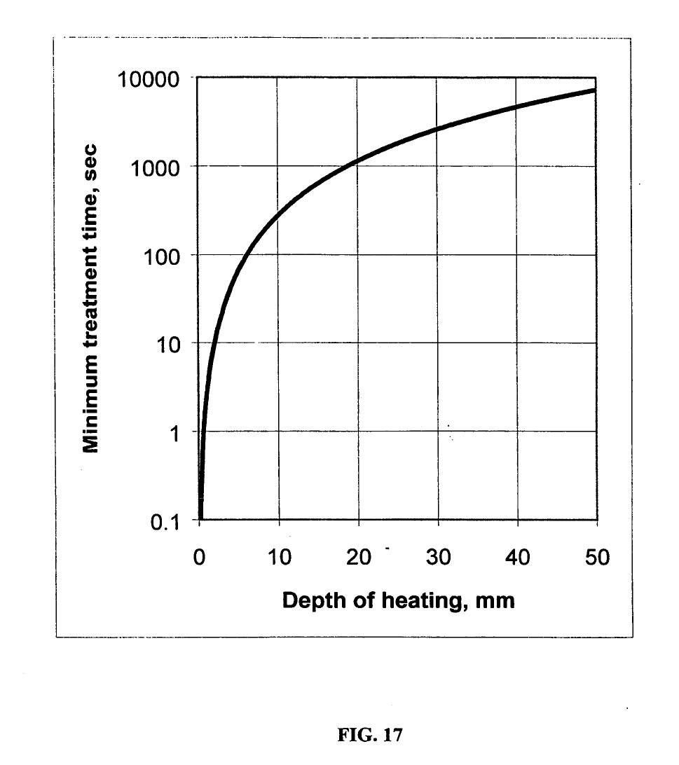

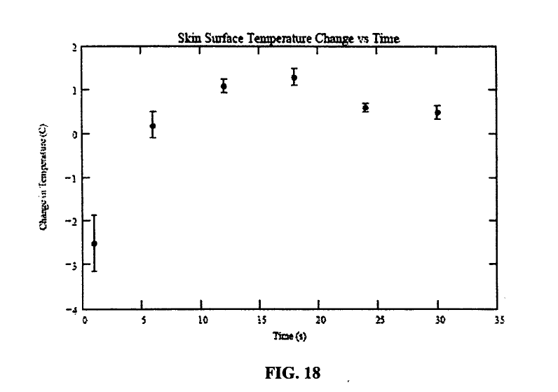

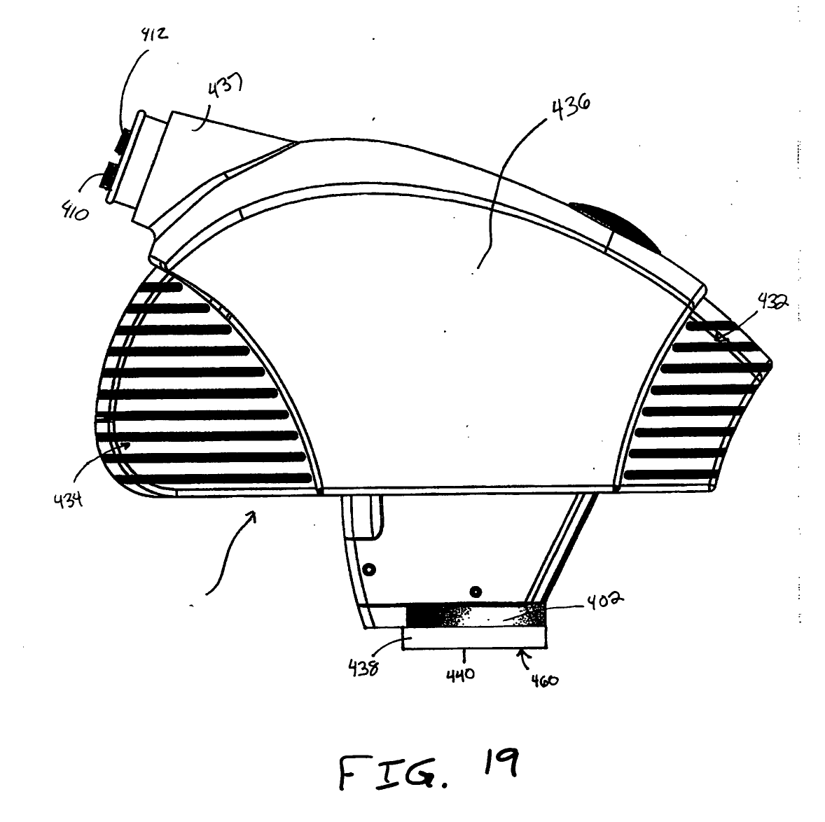

The graph in FIG. 17 illustrates the relationship between treatment time and depth of heating for light sources operating in the infrared wavelength. Although the depth of heating will be dependent on various factors, including the electromagnetic wavelength used, the type of tissue treated and the power density of the electromagnetic wavelength, FIG. 17 provides a general guideline of the parameters for heating tissue at depth using infrared wavelengths and power densities generally in the range of 0.5-5.0 W/cm². For comparison, the relationship between surface skin temperature (median and standard deviation) and treatment time when pre-cooling is used and the skin is continually cooled during treatment is shown in FIG. 18.

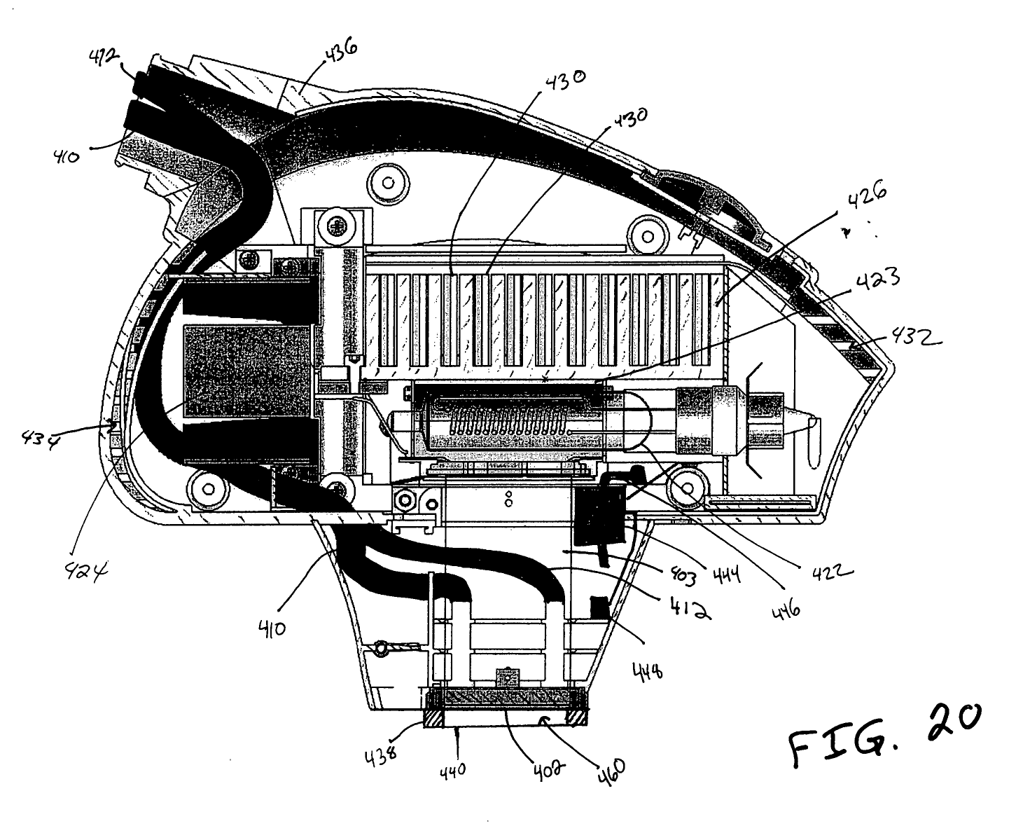

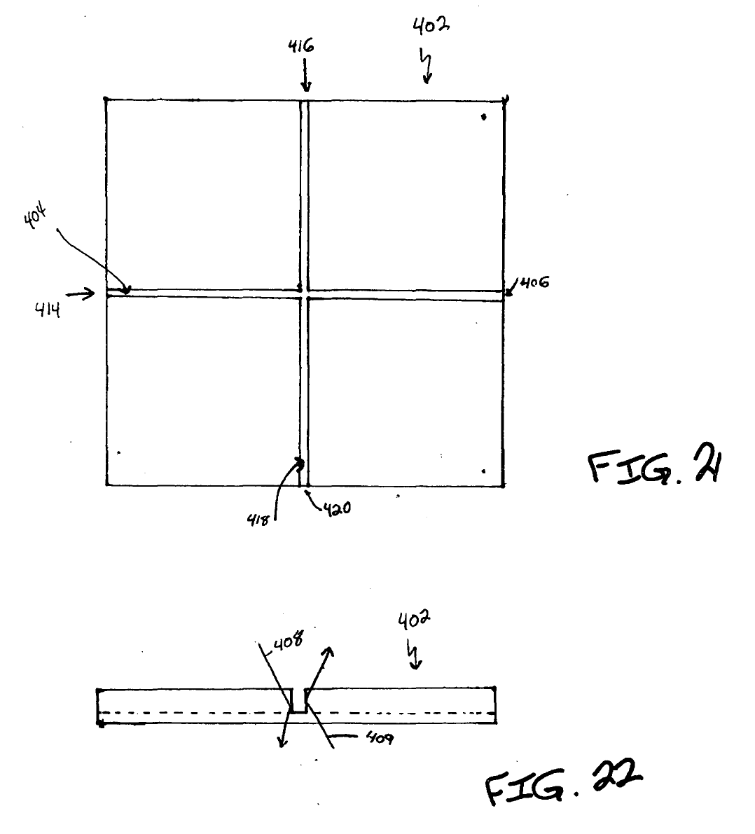

Referring to FIGS. 19 through 23, a handpiece 400 is capable of treating both the dermis and the fat or other tissue beneath the dermis. Alternatively, embodiments of the handpiece could be designed to heat tissue at relatively greater or shallower depths.

To heat tissue more deeply, whether using fractional or conventional methods, the handpiece 400 transmits light to the tissue at a relatively lower level of power for a longer period of time than prior art devices. In other words, the level of irradiance of the tissue is lower, but the power is delivered for a longer pulse width. For example, for some applications, such as collagen stimulation and certain types of pain relief, handpieces and other embodiments can be designed to deliver 10 W/cm² for a period of 1 to 10 seconds. To treat cellulite, however, lower power densities are preferred over a longer pulse width. Therefore, one embodiment of the handpiece 400 is designed to deliver 1-2 W/cm² over the same period of time or longer, that is preferably 0.5 s-600 s, although longer periods are possible, depending on depth and extent of treatment.

The following table provides preferable specifications for embodiments designed for several applications, although many other applications are possible.

TABLE 2
Specifications For Various Applications

| | Skin | | |
|---|---|---|---|
| Application | Remodeling | Acne | Cellulite |
| Spectrum of | 900-1350 nm | 900-1850 nm | 900-1350 nm |
| Wavelengths | | | |
| Window Size | 12 cm × 28 cm | 10 cm × 15 cm | 40 cm × 40 cm |
| Power Density | 50 W/cm² | 85 W/cm² | 1-4 W/cm² |
| Fluence | 5-240 J/cm² | 5-400 J/cm² | Up to 2500 J/cm² |
| Pulse Width | 0.25 10 sec | 0.25-5 sec | 0.5-600 sec |
| Skin Cooling | 5° C. | 5° C. | 5° C. |
| Temperature | | | |

In an alternate embodiment of the invention, devices such as devices 100 and 200 described above can be used to provide a lower power density by increasing the size of the window through which EMR is transmitted. In other words, rather than decreasing the power density by decreasing the relative amount of power that is produced by the device, the power density can be lowered by enlarging the area of the window that transmits energy to the tissue being treated. In addition to producing a desirable power density, increasing the area has the additional advantage of allowing the handpiece 400 to be used with the same base unit as other handpieces, such as the embodiments described in conjunction with FIGS. 1-15.

Furthermore, handpiece 400 also has the advantage of increasing the area of tissue that is treated at any one time, thereby making treatments faster and more efficient. Thus, the patient is required to spend relatively less time per visit and the person administering the treatment can perform relatively more treatments in the same amount of time.)

With the exception of the alternate window configuration and the inclusion of certain other additional features that are described below, handpiece 400 is essentially the same in function, structure and operation as devices 100 and 200 described above in association with FIGS. 1-16. By way of comparison, however, the devices described above include relatively smaller windows through which EMR passes. For example, referring to FIG. 14, device 200 includes a window 223 that is 12 mm by 28 mm and that allows light to be transmitted from lamp 202 (shown in FIGS. 8 and 9) to the tissue being treated. Such a rectangular shaped window can be cooled evenly and thoroughly, e.g., by flushing chilled coolant (generally water) along one or both of the longer 28 mm edges of the window, using the circulatory system discussed above. Such application of chilled coolant causes the heat to be evenly dissipated across the narrow span of the rectangular window.

On the other hand, referring also to FIGS. 19 to 23, the handpiece 400 has a relatively larger window 402 that, in this particular embodiment, is 40 mm by 40 mm. The larger window serves to reduce the power density to a level that is particularly suited to treat cellulite by increasing the area of the window relative to smaller windows while still using the same power supply and producing approximately the same amount of irradiance from the light source.

However, due to the large size of the window 402 in handpiece 400, passing fluid along one or more sides of the window is insufficient to dissipate heat from the center of the window, and a relatively hotter area will be created during operation of the handpiece 402, due to the buildup of heat in the center of the window. Therefore, additional features are provided to adequately cool the window, and eliminate any hot spot on the window during operation. In addition to providing cooling along the edges of the window, as in the device 200 and window 223, the window 402 includes two intersecting grooves 404 and 406 that are etched into the upper surface of the window 402. Additionally, window 402 is cooled on all four sides, while the window 223 is cooled only along the two longer sides.

The grooves 404 and 406 extend downward into the window 402 for a distance that is approximately two-thirds of the total thickness of the window. In this embodiment of the window, the grooves 404 and 406 are approximately 4 mm deep while the total thickness of the window 402 is approximately 6 mm, and the grooves are approximately 0.5 mm wide.

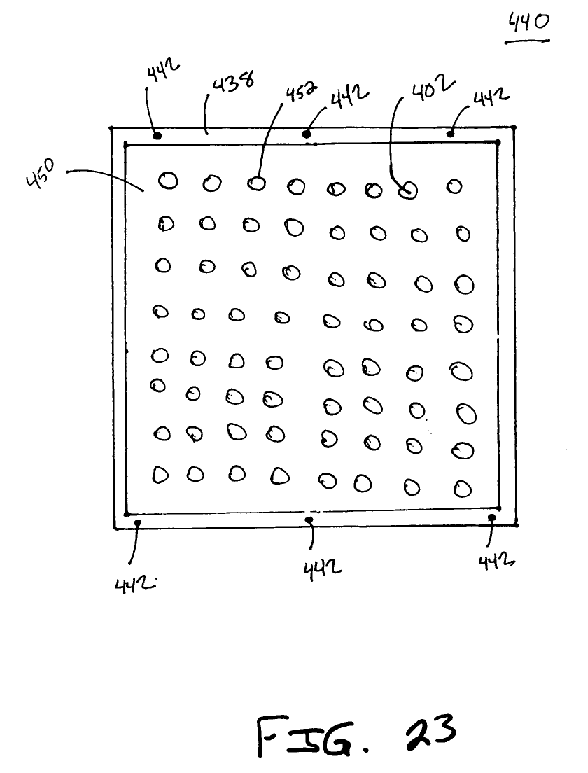

The configuration of the grooves 404 and 406 of the window provide sufficient cooling of the central portion of window 402 while obstructing only a minimal amount of light passing through the window during operation of the handpiece 400. First, due to the thin width of the grooves 404 and 406, the grooves 404 and 406 obstruct only a small portion of the window in the direction through which EMR passes. Second, due to the Total Internal Reflection (TIR) of light within the window against the walls of the grooves 404 and 406 as shown in FIG. 22, almost none of the light 408 or 409 that is incident upon the walls of the grooves 404 and 406 will pass into the grooves, whether the light is traveling from the handpiece or has been reflected back by the tissue. The same is true for light that is reflected or scattered back from the skin during use. The advantageous optical characteristics of the grooves 404 and 406 are due, in part to the relative disparity in the indexes of refraction of the material that forms the window 402 and the index of refraction of water.

Preferably, the grooves 404 and 406 are filled with water during operation. The index of refraction of water (which is approximately 1.33) is lower than the index of refraction of the sapphire window 402 (which is approximately 1.74). Therefore, as will be appreciated by one skilled in the art, light will have a tendency to be reflected by the boundary between the window 402 and the water due to the TIR. Only light that is incident against the boundary at very steep angles will pass through to the water. However, given the orientation of the light source to the window 402, almost all of the light will strike the boundary at an angle that will cause the light to be reflected off the boundary and to continue to pass through the window 402 to the tissue. Thus, only a small fraction of the light will pass into the grooves 404 and 406.

When the handpiece 400 is fully assembled, the upper surface of window 402 abuts a lower surface of a waveguide 403, essentially transforming grooves 406 and 408 into tunnels or capillaries through which cooling fluid can pass. The juncture between the waveguide and the sapphire window 402 preferably includes a dielectric coating that enhances the transmission of light from the waveguide 403 to the window 402 and also serves to seal the junction.

During operation, coolant, preferably chilled water flows from the circulatory system input tube 410 and into the groove circulatory inputs 414 and 416. The water, which has been chilled, preferably to approximately 5° C., flows through the grooves 404 and 406 and along all four sides of the window 402 to cool the window 402. The water passes through an intersection of the grooves 404 and 406 and continues to flow out of the groove circulatory outputs 418 and 420. At that point, the water, which is now relatively hotter due to the transfer of heat from the window 402 to the water, travels through the output tube and back to the chiller located in the base unit (not shown), where the water is cooled again and pumped back through the circulatory system.

It will be clear to one skilled in the art, however, that the parameters of the handpiece 400 can be altered to optimize the handpiece 400 for other applications. For example, many dimensions and shapes are possible in order to aid in the treatment of the tissue, cooling of the window, and/or for other reasons. Furthermore, a 40 mm by 40 mm window or other large size window could be used in a handpiece that produced light at relatively higher power levels to allow the handpiece to be used for treatments that require relatively higher power densities. Treatments such as hair removal that do not require heating tissue as deeply as cellulite and benefit from higher power densities could be performed using a relatively larger window similar to the window 402 of the handpiece 400. Use of such a handpiece would allow for hair removal treatments to be performed more quickly over larger areas of tissue, such as the back or legs. Additionally, the configuration of the grooves could be altered or additional grooves could be added to facilitate cooling of the window or to accommodate an even larger window. Also, hollow cuts, tunnels or capillaries could be created through a window to allow water to flow through the capillaries without having to abut the window against another object, such as a bottom surface of a waveguide, to provide a boundary across the top of a groove to contain the coolant. Additionally, the shape of the groove, cuts, tunnels or capillaries could be cut in various shapes, for example, with a “V” shape, in which the bottom of the “V” extends upwards in order to reduce or eliminate the passage of light through the flat portion of the grooves 404 and 406 that are largely perpendicular to the general direction of the EMR being irradiated. Again, the difference in the indexes of refraction of such a design would allow most of the light incident on the walls of the “V” portion to be reflected. The cuts may have circular, rectangular, triangular or other cross-section. The cuts may be distributed uniformly over the waveguide, thereby eliminating temperature gradients or at least decreasing the gradients from what they would be if only the sides are cooled. The cuts can be parallel or can intersect. The cooling may also be accomplished through evaporation of a liquid like Freon from the cut surfaces.

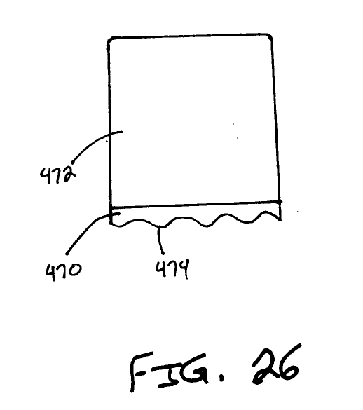

Similarly, as disclosed, window 402 is a monolithic plate, but it could also be composed of multiple pieces that are affixed together, e.g., glued together. However, in such an embodiment, the glue or binding material likely would absorb heat and, thus, decrease the thermal performance of the window. For comparison, referring to FIG. 25, an alternative method of cooling a window from the prior art is shown. A window 502 is cooled by providing a horizontal space 504 between two plates, e.g., sapphire window 502 and a quartz waveguide 506, thereby forming a continuous optical structure to transmit light or other EMR when water is passed through the space 504 to cool the window. However, in such an embodiment, some of the light would be reflected back toward the light source at the interfaces between the water channel and the waveguide and the water channel and the window and the water would absorb some of the energy passing through the window.

Referring to FIGS. 19 and 20, handpiece 400 includes two cooling circuits, each particularly adapted to its purpose. The first cooling circuit cools a contact surface of the handpiece in order to cool the tissue being treated and the second cooling circuit cools the light source. The handpiece 400 is configured to irradiate tissue using near infrared EMR, and it includes a circulatory system to remove heat from the surface of the tissue to be treated and thereby cool the skin and a fan system to cool the infrared lamp. The circulatory system allows chilled fluid, typically water that is chilled to approximately 5° C., to flow from a base unit (not shown), into the handpiece 400 through input tubing 410, around the cooling window 402, and out of the handpiece 400 through output tubing 412. The cooling window 402 can be made of various suitable materials, but is preferably sapphire in the present embodiment.

In the apparatus proposed, skin cooling is implemented through contact with the cooled tip of the sapphire window 402. Several mechanisms for cooling the window 402 are possible. For example, the window should be of a material having good thermal conduction properties, such as sapphire, and cooling fluid can run along one or more of the edges of the window and/or the window can have a plurality of hollow cuts or capillaries extending through the window, with cooling liquid, preferably chilled water, or gas circulating through the cuts, as described above.

The handpiece 400 also includes a second cooling circuit to remove heat generated by light source 422. Light source 422 is a halogen lamp that is designed to operate at a high temperature. The bulb of halogen lamp will be approximately 500° C. during operation, and relatively little heat energy must be removed to keep the light source 422 within operating limits and prevent overheating. Further, because halogen lamps work more efficiently as the temperature increases, removing too much heat from around halogen lamp 400 may reduce the efficiency of the lamp and the performance of handpiece 400. Thus, light source 422 can be cooled with a second circulatory system that does not require an additional cooling mechanism, such as a chiller. Instead, a simpler and less expensive air cooling system can be used.

In similar prior art handpieces, a single cooling circuit is used to cool both the tissue contacting surface and the light source. Using a single cooling circuit means that a compromise must be made between cooling the light source which, as indicated above, runs at a very high temperature, and cooling the skin which is maintained at a much lower temperature to prevent injury. For example, one prior art device compromises by using a single cooling circuit to cool both the light source and skin contact surface to 20° C. Cooling the lamp to 20° C. puts a very large burden on the chiller and also does not allow the lamp to run at the more efficient higher temperature. Cooling the skin contact surface and, thus, the skin, to only 20° C. limits the amount of light that can be applied to the skin without injury.

Using the first and second cooling circuits as described above eliminates the need for this compromise. The lamp can run at the much higher and more efficient temperature of, for example, 500° C., and be cooled with only a simple, small, inexpensive cooling circuit, such as one or more fans, while the skin contacting surface can be cooled to much lower temperatures, for example, 5° C. or lower, allowing more light to be applied to the skin without injury. As a result, the cooling capacity of the water from the chiller located in the base unit is not unnecessarily utilized to cool the lamp. This reduces the burden on the chiller and has the additional advantage of allowing the chiller to be smaller and less expensive or allowing the same size cooler to cool the skin contacting surface to a lower temperature.

Preferably, for devices utilizing halogen lamps, the lamp is coated or otherwise surrounded with a highly reflective material, which increases the efficiency of the lamp. Such an arrangement is disclosed in a U.S. patent application entitled “LAMP FOR USE IN A TISSUE TREATMENT DEVICE” filed Feb. 17, 2006 and assigned to Palomar Medical Technologies, Inc.)

In the present embodiment, a fan unit 424 cools the light source, which includes a lamp 422, a reflector 423 and a heatsink 426. Fan unit 424 pumps air into the handpiece 400 and across heatsink 426, which is attached to the top of lamp reflector 428 to allow heat to be transmitted from the reflector to the heatsink. Reflector 428 is preferably coated with gold or other highly reflective metal, such as silver or copper. The heatsink 426 includes fins 430 that dissipate heat to the air, as the air flows around the fins 430 and, subsequently, exits the handpiece 400. The air enters and exits the handpiece 400 via vents 432 and 434 respectively, which are located on opposite ends of the handpiece and are formed as an integrated part of a housing 436 of handpiece 400.

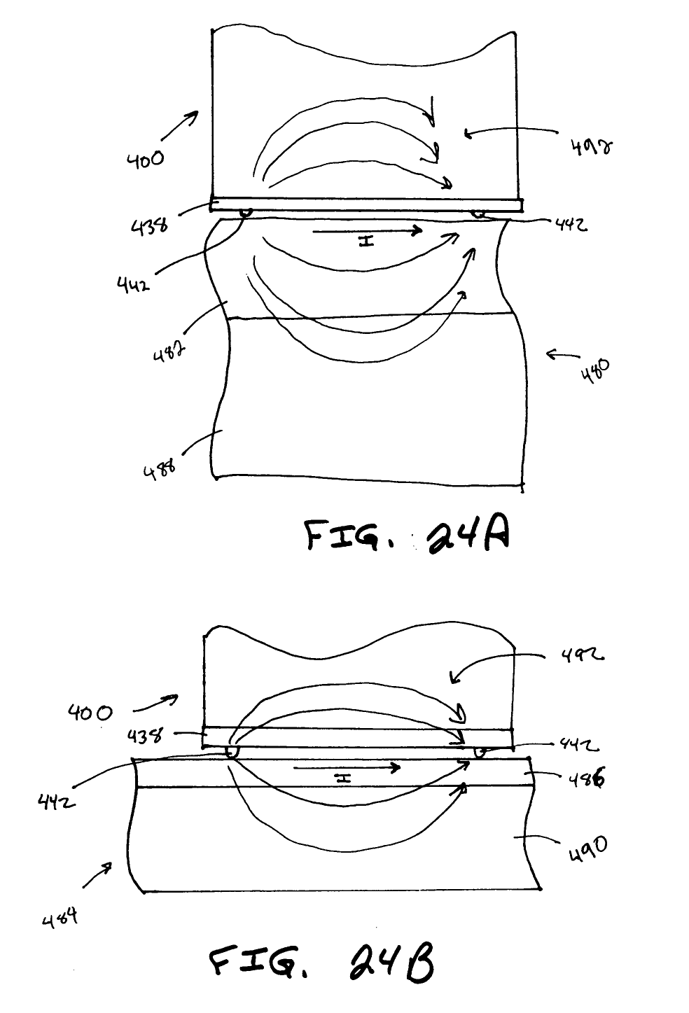

In some embodiments, a mask can be used to block portions of the EMR generated by the EMR source from reaching the tissue. The mask can contain a number of holes, lines, or slits, which function to spatially modulate the EMR to create islets of treatment. FIG. 23 illustrates an embodiment in which the islets of treatment are formed generally through the use of a mirror containing openings 452 that are small holes.

Referring to FIGS. 20 and 23, the handpiece 400 transfers light to the tissue being treated through the sapphire window 402 located in the face 440 of the handpiece 400. The window 402 is adapted for fractional treatments and, therefore, includes a mask 450 having an array of relatively small circular openings 452, while the remainder of the mask covering the window 402 is opaque and does not pass EMR of other wavelengths during operation. Although the mask may pass some EMR, substantially more will pass through the openings 452. (As discussed below, other embodiments could be adapted for non-fractional applications.) In one embodiment, the mask 450 consists of carbon particles in a film, which is placed in contact with the surface of the skin. The mask 450 is attached to the sapphire window 402, and the mask 450 is positioned between the optical energy source, here lamp 422, and the target tissue when the apparatus is in use. The mask 450 may instead include one or more dielectric layers with a plurality of openings 452 for passage of EMR from the lamp 422 to the target area. Handpiece 400 can, therefore, create treatment islets in the patient's skin. Other embodiments of dermatological devices having similar masks are disclosed in U.S. Patent Application No. 60/561,052, entitled Methods and Products for Producing Lattices of EMR-Treated Islets in Tissues, and Uses Therefore and filed Apr. 1, 2005, which is incorporated herein by reference.

Light passes through the openings 452 in the mirror and strikes the patient's skin, creating islets of treatment. Light reflected by the mirror stays in the optical system through a system of reflectors and may be redirected through the holes to improve efficiency. One effective mask is a contact cooling mask (i.e., it contacts the skin during treatment) with a high reflection and minimum absorption for masking light.

In this aspect, the dielectric layers can have a high reflectance over a spectral band emitted by the lamp 422. The openings in the mask 450 can have various shapes or identical shapes. For instance, the openings can be lines, circles, slits, rectangles, ovals, or irregular shapes. In some aspects, the apparatus can include a cooling or a heating element for cooling or heating the mask during use. The optical energy can be over a wide wavelength band, and, in this case, infrared light is used. The optical energy can be applied with various pulse widths, preferably 100 msec to 1 sec.

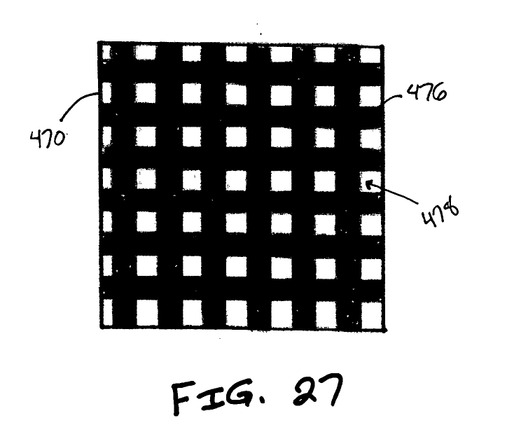

Similarly, referring to FIG. 26, other configurations of the face of the handpiece are possible. For example, the window 470 attached to a waveguide 472 may have spatial non-uniformities. In this case, damage of the skin will be non-uniform. The size of the non-uniform fields may be less than 50 μm. The non-uniform damage may be useful for skin rejuvenation, or for vascular or pigmented lesions, tattoos, etc., because it decreases the peak of extremely strong damage of the skin: blistering, purpura etc. At the same time, the damaged islands heal quickly because tissue between the damaged islands is not damaged and can therefore provide cell proliferation.

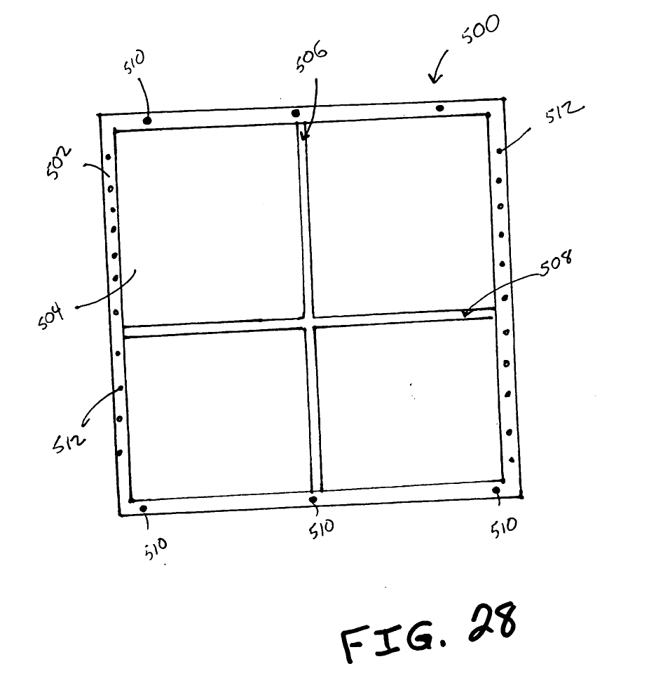

In order to provide non-uniform damage of the skin surface, the window 470 of the waveguide may have a modulated profile 474 as is shown in FIG. 26. A spatial mask 476 may also be coated (reflected mask) on the front surface of the window 470, for example a flat mask having square openings 478 as shown in FIG. 27. Patterned index variations (phase mask) in the waveguide may also be employed. Other optical techniques may also be utilized to accomplish this objective. At least some of the techniques indicated redistribute light to provide selected treatment spots.

Referring again to FIGS. 20 and 23, a face 440 of handpiece 400 further includes proximity sensors 442 that are located about the perimeter of the window 402. The sensors can be aligned as shown in FIG. 23, or alternatively, many other embodiments are possible, including placing sensors on each side of a window, on adjacent sides of a window, at the corners of the window, or in various combinations of these or other configurations. During operation, the sensors 442 ensure that the face of the handpiece 400 is in close proximity to or in contact with the skin or other tissue before the handpiece 400 can be “fired,” i.e., engaged to cause light to be emitted by the lamp 422 and from the handpiece 400. The proximity sensors 442 can be any of a number of appropriate sensors, including pressure sensors, similar in function to the sensor described in conjunction with device 200 that ensure that the handpiece 400 is actually in contact with and pressed against the tissue before the handpiece 400 can be fired.

In the present embodiment, however, electrical field sensors (also know as e-field sensors) are preferred. The e-field sensors 442 detect changes in a low-level electrical field when, e.g., a portion of tissue enters the field. Therefore, the sensor can be used to detect when the tissue is in close proximity to the sensors. Because the sensors are located on the face of the handpiece 400, and about the sapphire window 402, the sensors are able to detect when the tissue is in close proximity to or in contact with the sapphire window 402, and are used to determine when the tissue is in a suitable position for firing the handpiece 400.

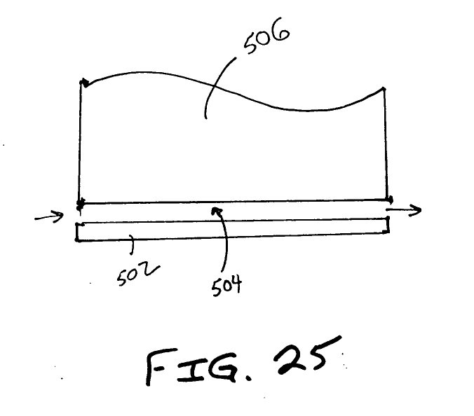

Referring to FIGS. 24A and 24B, the e-field sensors can also be used as sensors to determine the type of tissue that is in close proximity to the window 402. The underlying composition of tissue varies based on its location on the body. For example, normal skin tissue 480 has a relatively thicker dermal layer 482 than tissue 484 near the eye, which has a relatively thinner dermal layer 486. Similarly, normal skin tissue 480 has a relatively thicker layer of fat 488 underneath the dermis 482, while the tissue 490 around the eye at similar depths is mostly water. The different compositions of the tissue will affect an electrical field 492 of an e-field sensor differently. The e-field sensors 442 can detect these different effects to differentiate between, e.g., normal skin tissue and tissue located over or near the eye, or to differentiate other types of tissue. The proximity sensors 442, therefore, can be used to provide additional features, such as safety features. For example, if the proximity sensors 442 detect that the face of the handpiece 400 is in close proximity to skin over or near the eye, the controller can cause the handpiece 400 to stop operation or operate with a lower level of irradiance to protect the eye. Similarly, the controller can cause the handpiece 400 to provide various intensities or wavelengths of light for various tissue types to optimize the treatment being provided.

Alternatively, other sensors could be used to provide contact sensing as well as other features. For example, two electrical contacts could be located in the portion of the handpiece 400 in contact with the skin. When the resistance (or capacitance) measured between the two contact elements was within a range typical for skin, the laser would be enabled to fire. It may also be possible to use a magnetic sensor to detect skin/sapphire contact. Similarly, a capacitive sensor could be used in conjunction with image processing to allow for determination of whether the device is operating on biological skin or some form of other surface. It is possible under proper sampling conditions to extract the type of skin the device is located above. This is accomplished by comparing real time processed images to a stored pattern or calculated set of parameters. In addition, the combination of the capacitive sensor and image pattern recognition, navigation algorithm, and special lotion, can be used to determine the presence of skin hair and provide statistical information about the density and size of the hair.

Handpieces preferably include sensors to make them both eye and skin safe. Many of the applications discussed above require high optical power (˜80-500 W), and a reliable contact sensor is typically used to enable the laser to fire only when the optical system (e.g., a sapphire element) is in good contact with the skin. For example, an embodiment of an apparatus to determine contact would include a small illumination source (e.g., diode laser or LED) mounted a few mm away from the window through which EMR passes (e.g., a sapphire element). The laser or diode is preferably located inside the device near the window 402. An illumination source is aimed at the skin surface and may emit at a different wavelength than the high-power light source. A detector having a filter to eliminate light at the treatment wavelength would be located in the handpiece to detect light from the illumination source that has been reflected or scattered from the skin. Thus, when the sapphire is in good contact with the skin surface, scattering and absorption in the skin would attenuate light from the illumination laser. In the case of poor or no skin contact, light from the illumination laser would propagate through the optical system to the detector. Thus, by setting an appropriate threshold, the laser could be configured to fire only when the detector is below a preset level. Note that such a detector could also be located in the base unit and an optical fiber used to couple light from the handpiece to the detector.

A second exemplary embodiment of an apparatus for determining optical contact eliminates the use of an illumination source. In this case the detector is configured to detect only light from the treatment source by placing a bandpass filter in front of the detector. This method preferably activates the treatment source in a low-power eye-safe mode until firm contact with the skin is made. Thus, when there is no or poor contact between skin and handpiece, the detector output is relatively low. However, when the optical system (e.g., a sapphire element) is in good contact with the skin, the detector output will be relatively high. Thus, the treatment source would only fire when the detector output was above a preset threshold level.

A simple mechanical sensor could also be used to detect skin/sapphire contact. A spring-loaded pin that was depressed upon contact could be used to enable the laser. Multiple pins located around the perimeter of the sapphire could be used to ensure that the entire sapphire face was in good contact with skin. A commercially available load cell could also be used as a contact sensor.

Typical skin surface temperature is in the 30-32° C., and a temperature sensor could be used to detect skin contact. If the location in which the device was used was with the standard 23-27° C. range, the light source could be enabled when the temperature measured by the sensor was within the appropriate range. Alternatively, the laser could be enabled only when the proper temperature versus time slope was measured and disabled when the measured temperature was outside the desired range.

Contact sensor design is described in greater detail in U.S. application Ser. No. 09/847,043, by Henry Zenzie, filed Apr. 30, 2001, entitled “Contact Detecting Method and Apparatus for an Optical Radiation Handpiece,” the substance of which is hereby incorporated by reference.

Referring to FIGS. 19 to 23, handpiece 400 has additional features to assist in the treatment of tissue. For example, the handpiece 400 includes a frame 438 about the window 402. The frame is 50 mm by 50 mm on the outer edge, and has a width of 5 mm and a thickness of 8 mm. The frame is made of plastic. The junction between the frame and the face of the handpiece 400 is airtight. In the present embodiment, the frame 438 is a separate piece that is attached to the face using screws and a sealant. In other embodiments, the frame could be, e.g., formed as an integral part of a handpiece as an injection molded plastic or other material.

The handpiece 400 further includes a pump 444, a connection tube 446 and a pressure switch 448.

During the operation of the handpiece 400, the frame 438 is placed against the tissue such that an area of tissue to be treated lies within an area bounded by the frame 438. The pump 444 evacuates air from the volume of space 460 bounded by the window 402, the frame and the tissue through the connection tube. Thus, the pump 444 creates a vacuum, which, in turn, causes the tissue to be pulled into the evacuated space. Preferably the tissue is pulled against the window 402 of the handpiece 400. During operation, the pressure in the space 460 bounded by the tissue, the frame 438 and the window 402 is 15 in Hg and forms a vacuum.

The pressure switch 448 is connected to the pump 444 via a wire. Both are connected to a controller (not shown) in the base unit that receives inputs from pressure switch 448 and controls pump 444 via an umbilical chord that attaches to handpiece 400 at connector 437. During operation, the pressure switch 448 ensures that the skin remains in contact with the handpiece 400 during treatment. Preferably, the area of tissue being treated will remain in contact with the window 402, but may be treated even when not in direct contact with the window 402. If the contact between the tissue and the frame 438 is broken or compromised, air will enter the previously-evacuated space and cause a change in pressure. The pressure switch 448 will sense the change in pressure and send a signal to the controller in the base unit that causes the controller to stop the operation of handpiece 400. When that happens, the handpiece 400 can also provide an alarm to the operator to notify the operator that the contact between the skin and the handpiece 400 has been compromised and/or is not complete. The pressure switch 448 is configured to send a signal indicating that the contact is incomplete. The alarm can be communicated to the operator by one or more of a number of notifications, including without limitation, a flashing light, a sound, or the display of an error code or other information.

The use of suction to pull the area of tissue being treated against (or in close proximity to) the window 402 of the handpiece 400, is thought to have several advantages, such as the maintenance of good contact between the tissue and the handpiece 400 during treatment. For example, if a handpiece relies on the operator to apply pressure to make contact between the tissue and the handpiece during treatment, the system may allow the operator to treat tissue even when the contact is not optimal, such as when pressure is applied unevenly and/or the entire window 402 of the handpiece 400 is not in complete contact with the tissue during treatment.

The use of suction to provide contact also may have the benefit of increasing blood flow to the skin by distending the tissue, and the blood vessels within the tissue. An increase in blood flow within the tissue being treated will assist in the cooling of the skin at the surface, as the additional blood flowing through the tissue during treatment will provide additional heat capacity, and the blood will carry heat from the tissue as it circulates through the circulatory system of the person being treated.

The handpiece can be further combined to provide for additional types of stimulation intended to enhance the treatment of the tissue. For example, the muscles in the tissue, such as facial muscles, can be stimulated to induce muscle contraction during the treatment. Referring to FIG. 28, in an alternate embodiment of a window assembly 500 that is suitable for use with the handpiece 400. Window assembly 500 includes a frame 502 about a window 504. Window 504 is similar in structure to window 402, having intersecting channels 506 and 508. In this embodiment, window 504 does not have a mask attached or applied, although such a mask could be included in other embodiments. A set of contact sensors 510 are disposed about two opposing sides of the frame 502, while a set of electrical pins 512 are provided along the other two sides of the frame 502. The electrical pins 512 allow for electrical stimulation of the muscle tissue. An electrical current is applied to the tissue via the electrical pins 512, which causes a contraction of the underlying muscles.

Similarly, a piezoelectric motor or a DC motor could be included to provide for vibration of the tissue during treatment. Such additional features are thought to enhance the treatment of the tissue.

While several embodiments of the invention have been described and illustrated herein, those of ordinary skill in the art will readily envision a variety of other means and structures for performing the functions and/or obtaining the results and/or advantages described herein, and each of such variations or modifications is deemed to be within the scope of the present invention.

For example, those skilled in the art will appreciate that while embodiments have been described in the context of handpieces that can be used interchangeably with a base unit, many other embodiments are possible. For example, a single device could incorporate the base unit and one or more handpieces as a solitary system. Additionally, devices other than handpieces are possible. For example, where applications require longer treatment pulses or longer treatment times to achieve deep heating of tissue, devices that are not required to be held during operation would be advantageous. Thus, a device intended to treat one area of tissue for an extended period could be configured in the form of a pressure cuff or a stationary heating pad that could be laid, taped, clipped, strapped, etc. to the person being treated.

More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. The present invention is directed to each individual feature, system, material and/or method described herein. In addition, any combination of two or more such features, systems, materials and/or methods, if such features, systems, materials and/or methods are not mutually inconsistent, is included within the scope of the present invention.

1. A window of a dermatological treatment device configured to transmit EMR from a source for generating EMR to tissue being treated, the window comprising:
a pane configured to allow EMR to pass from the dermatological treatment device to the tissue being treated; and
at least one cooling channel extending across a portion of the pane, wherein the area of the channel is substantially less than the area of the pane.

2. The window of claim 1 further comprising:
a frame extending about the pane to secure the pane in the dermatological treatment device;
a first cooling input in fluid communication with a first end of the first channel;
a first cooling output in fluid communication with a second end of the first channel; and
wherein the window is configured to be cooled during operation by fluid traveling through the cooling input, through the first channel and out the second end of the first channel.

3. The window of claim 1, wherein the at least one channel is a groove having an open portion extending along a surface of the pane, and wherein the window further includes an optical surface abutting the surface of the pane such that the groove is enclosed during operation to allow fluid to flow through the channel and to prevent the fluid from flowing out of the open portion.

4. The window of claim 1, wherein the window further includes an optical material between the pane and the optical surface, wherein the material allows some EMR to pass from the dermatological treatment device to the tissue being treated.

5. The window of claim 4, wherein the material is a dielectric coating.

6. A dermatological treatment device for treating tissue located at a depth of at least approximately 0.5 mm, comprising:
a housing containing an EMR source and a window configured to transmit EMR from the source to the tissue being treated;
wherein said power source is configured to produce at least 500 W and the window has an area sufficiently large to produce a power density of less than 5 W/cm².

7. The dermatological treatment device of claim 6, wherein the pulse width of the power source is greater than or equal to 0.5 seconds.

8. The dermatological treatment device of claim 6, wherein the pulse width of the power source is between 0.5 seconds and 600 s inclusive.

9. The dermatological treatment device of claim 6, wherein the EMR source is configured to produce at least 1000 W.

10. A dermatological treatment device configured to transmit EMR to tissue being treated, the device comprising:
a housing containing a source configured to emit EMR and a treatment window configured to pass EMR emitted by said source to said tissue;
wherein said window has a tissue contact surface area that is greater than 600 cm².

11. The dermatological treatment device of claim 10, wherein the window includes:
a pane configured to allow EMR to pass from the dermatological treatment device to the tissue being treated, at least one cooling channel extending across a portion of the pane, wherein the area of the channel is substantially less than the area of the pane.

12. A window of a dermatological treatment device configured to transmit EMR from a source for generating EMR to tissue being treated, the window comprising:
a pane configured to allow EMR to pass from the dermatological treatment device to the tissue being treated; and at least one cooling channel extending across a portion of the pane, wherein the area of the channel is substantially less than the area of the pane.
